(19) United States
(12) Patent Application Publication
(10) Pub. No.: US 2019/0240335 A1
(43) Pub. Date: Aug. 8, 2019

(54) Biocompatible Hydrogel Compositions

(71) Applicant: Promedon S.A., Cordoba (AR)

(72) Inventors: Karina Bierbrauer, Cordoba (AR); Roxana V. Alasino, Cordoba (AR); Dante M. Beltramo, Ciudad de Cordoba (AR); Osvaldo N. Griguol, Cordoba (AR)

(21) Appl. No.: 15/891,129

(22) Filed: Feb. 7, 2018

(51) Int. Cl. A61K 47/38; A61K 9/00; A61K 47/69; A61K 31/737; A61P 19/02; C08B 37/00; C08B 15/00; C08B 15/06; C08J 3/24; C08J 3/075

(52) U.S. Cl. CPC A61K 47/38; A61K 9/0024; A61K 47/6903; A61K 31/737; A61P 19/02; C08J 2301/02; C08B 15/005; C08B 15/06; C08J 3/246; C08J 3/075; C08J 2305/00; C08B 37/0069

(57) ABSTRACT

The present disclosure encompasses biocompatible hydrogel compositions comprising covalently bonded hydrogel reaction products, as well as compositions of, methods of producing, methods of using, and kits comprising the covalently cross-linked hydrogel reaction products. The covalently cross-linked hydrogel reaction products can be derived from a cross-linking reaction of a polyquaternium-10, a chondroitin 4-sulfate, and a divinylsulfone.

### TECHNICAL FIELD

The present disclosure relates generally to biocompatible hydrogels and, more particularly, to biocompatible hydrogels compositions comprising reaction products of a cationic cellulose-derived polymer covalently bonded with a naturally derived anionic polymer and uses and methods of making same.

### BACKGROUND

Osteoarthritis is a disease that involves the whole articulation of a joint. Among the articular structural damage, it is possible to observe the change of the synovial liquid properties. Several methods of treatment of osteoarthritis have been attempted, from dietary supplements to viscosupplementation with implantable corticoids and polymers and even surgical procedures. However, each attempted treatment has drawbacks that necessitate the need for further improvement.

The implantation of biocompatible polymeric materials and gels to treat the condition often results in the need for repeated and frequent injections over the course of time due to the rapid breakdown of the implanted material. Like osteoarthritis, other conditions are treated with the implantation of biocompatible polymeric gels. In each case, the rapid breakdown of the biocompatible polymeric gel limits the efficacy of the treatment and sometimes requires repeated injections to produce noticeable results. Indeed, biocompatible polymeric gels can be used in a variety of applications, but in many such cases shortfalls in the stability of the biocompatible polymeric gels limit their utility and efficacy.

Consequently, there is a need for a biocompatible polymeric material or gel that can address one or more of the deficiencies in the currently available materials.

The article entitled, “Cationic Cellulose And Its Interaction With Chondroitin Sulfate. Rheological Properties of the Polyelectrolyte Complex”, describes “the polyelectrolyte complexation between polyquaternium cellulose (polyquaternium-10) and chondroitin 4-sulfate (C-4S) . . . . The complex shows different behaviors with increasing concentrations of the polyanion. Initially, polyquaternium-10/C-4S interaction forms a soluble, translucent hydrogel that reaches maximum viscosity at a sulfate carboxylate/quaternary ammonium molar ratio lower than 1. After that, the complex begins to aggregate until complete precipitation, and finally, at higher concentrations of C-4S, the resuspension of the aggregate is observed. Further addition of polyquaternium-10 initiates new cycles of precipitation/resuspension for at least seven times. C-4S contains sulfates and carboxylates able to interact with quaternary ammonium of polyquaternium-10.”

U.S. Pat. No. 4,582,865 is directed to cross-linked gels of hyaluronic acid, alone or mixed with other hydrophilic polymers and containing various substances or covalently bonded low molecular weight substances and processes for preparing them.

U.S. Pat. Nos. 4,767,463 and 4,913,743 are directed to combinations of glycosaminoglycan and certain cationic polymers to provide modified glycosaminoglycan properties and can provide substantivity to keratinous material, compatibility, stability, humectancy, rheology and other properties useful in personal care or medical applications.

U.S. Patent Application No. 2003/0086899 A1 is directed to viscoelastic compositions and methods of their use in treating joints, especially in conjunction with trauma and osteoarthritis.

European Patent No. 22272297 is directed to a cross-linked derivative of hyaluronic acid partially N deacetylated, comprising at least one repeating unit of formula wherein R1 is H or a C1-C20, substituted or unsubstituted moiety, derivative of an aldehyde of the aliphatic, aromatic, arylaliphatic, cycloaliphatic, heterocyclic series; R2 is an aliphatic, aromatic, arylaliphatic, cycloaliphatic or heterocyclic group substituted or unsubstituted; R is OH, o-, an alcohol group of the aliphatic, aromatic, arylaliphatic series, cycloaliphatic, heterocyclic, or an amino group of the aliphatic, aromatic, araliphatic, cycloaliphatic, heterocyclic series; the R3 groups, the same or different from each other are H, SO₃⁻ or a residue of the hemiesters of succinic acid or of heavy metal salts of succinic acid hemiesters; and wherein the R4 groups, the same or different from each other, are a COR group, or a CH₂OR₃ group.

U.S. Patent Application No. 2005/0069572 A1 is directed to a multi-layered tissue construct that includes: a first layer comprising a first hydrogel; and a second layer comprising a second hydrogel, wherein the first layer is connected to the second layer at a first transition zone and wherein at least one of the first layer and the second layer further comprises a component selected from the group consisting of cells and a bioactive substance. Another multi-layered tissue construct includes: a first layer comprising a first hydrogel; a second layer comprising cells of a first type, wherein the second layer is disposed on the first layer; and a third layer comprising a second hydrogel and optionally cells of the first type encapsulated in the second hydrogel, wherein the third layer is disposed on the second layer. Methods for producing these multi-layered tissue constructs are also disclosed.

U.S. Pat. No. 8,574,620 B2 is to directed to a biocompatible composite and method for its use in repairing tissue defects, including defects in cartilage. The biocompatible composite includes a fibrous polymeric component and a polymerizable agent, which is capable of forming the biocompatible composite in situ at the site of a tissue defect. In embodiments, the repair site at which the biocompatible composite is to be applied may be treated with a priming agent, permitting polymerization of the polymerizable agent to the tissue located at the repair site.

U.S. Pat. No. 9,050,392 B2 is directed to a method for preparing a cross-linked sterile and homogeneous hydrogel for injection, characterized in that it comprises the following steps: (a) preparing an aqueous solution containing a polymer derived from cellulose and at least one water-soluble polymer, the total polymer content ranging from 0.5 and 5 wt %, preferably from 1 to 4 wt % and more preferably from 1.5 to 3 wt %; (b) optionally adding solid particles; (c) pouring the resulting liquid mixture with the optional solid particles into a vessel and closing dais vessel using a water-tight and gas-tight system; and (d) exposing said vessel containing the liquid and the optional solid particles to a radiation dose of between 5 and 50 kGy, preferably between 20 and 30 kGy, and more preferably of about 25 kGy. A hydrogel obtained according to the above method and to the use thereof in medical applications is also disclosed.

U.S. Patent Application No. 2016/0303281 A1 is directed to water-insoluble but water-swellable and deformable cross-linked PEGylated microgel particles of proteins and protein-based macromolecules that are pseudoplastic (shear thinning) and flow in aqueous media under shear and which can be injected or made to flow, wherein said microgel particles can reform as a duster of microgel particles when shearing forces are removed. The microgel particles function as a matrix to support cell growth, viability, and proliferation.

The article entitled, “Chitosan-Chondroitin Sulfate And Chitosan-Hylanurate Polyelectolyte Complexes. Physico-Chemical Aspects” describes “fully deacetylated chitosan . . . contacted in solution with chondroitin sulfates and hyaluronic acid . . . pure polyelectrolyte complexes are formed.”

The article entitled, “Divinylsulphone-Activated Agarose Formation Of Stable And Non-leaking Affinity Matrices By Immobilization Of Immunoglobulins And Other Proteins” describes, “divinylsulphone-activated agarose is an attractive alternative to several activated supports usually used.”

The article entitled, “Ellipsometry Studies Of The Mucoadhesion Of Cellulose Derivative” describes “The mucoadhesion of three different cellulose derivatives, ethyl(hydroxyethyl)cellulose (EHEC), hydrophobically modified hydroxyethyl cellulose (h-HEC) and a cationic cellulose derivative (ammo cellulose), was investigated by in situ ellipsometry.”

The article entitled, “Interactions Of Ibuprofen With Cationic Polysaccharides In Aqueous Dispersions And Hydrogels Rheological And Diffusional Implications” describes “the association processes of sodium ibuprofen with cationic celluloses (CELQUAT® H-100 (PQ-4) and SC-230M (polyquaternium-10)) and cationic guar gums (ECOPOL® 261-S and 14-S) and their repercussions on the properties of the aqueous dispersions and cross-linked hydrogels.”

The article entitled, “Cationic Cellulose Hydrogels: Kinetichondroitin 4-sulfate Of The Cross-linking Process And Characterization As pH-/ion-sensitive Drug Delivery Systems”, describes “cross-linking process of two cationic hydroxyethylcelluloses of different hydroxyethyl and ammonium group contents, polyquaternium-4 (PQ-4) and polyquaternium-10 (polyquaternium-10), with ethylenglycol diglycidylether (EGDE) was characterized and optimized through rheometric analysis of the forming network. The influence of NaOH concentration, temperature, and EGDE concentration on the cross-linking rate were studied.”

The article entitled, “Effect Of The Counterion Behavior On The Frictional-Compressive Properties Of Chondroitin Sulfate Solutions”, describes “the thermodynamic response of aqueous chondroitin sulfate solutions to changes in the monomer and added salt concentrations, using a recently developed field-theoretic approach beyond the mean field (MF) level of approximation.”

The article entitled, “New Cationic Hydrophilic And Amphilic Polysaccharides Synthesized By One Pot Procedure” describes “synthesis of cationic polysaccharides carrying quaternary ammonium groups of various chemical structures . . . performed by one pot procedure involving the chemical modification of a neutral polysaccharide (dextran, pullulan) with an equimolar mixture epichlorohydrin/tertiary amine, in aqueous media.”

The article entitled, “Efficacy And Safety Of A Single Intra-articular Injection Of 2% Hyaluronic Acid And Mannitol In Knee Osteoarthritits Over A 6-month Period”, describes the evaluation of a single intra-articular injection of 2% hyaluronic acid (HA) and mannitol in symptomatic knee osteoarthritis (KOA).

The article entitled, “Ocular Biocompatibility Of Polyquaternium 10 Gel: Functional And Morphological Results” describes the “study of topical and intraocular biocompatibility and toxicity of cationic hydroxyethylcellulose Polyquaternium 10 (polyquaternium-10). It also evaluates the rheological properties of gels.”

### SUMMARY

The present disclosure encompasses biocompatible hydrogel compositions comprising covalently cross-linked hydrogel reaction products. The present disclosure encompasses a composition comprising: a covalently cross-linked hydrogel reaction product of a cross-linking reaction of a reaction mixture comprising a polyquaternium-10, a chondroitin 4-sulfate, and a divinylsulfone, and wherein the covalently cross-linked hydrogel reaction product exhibits a gel content in the range of 65% to 85% as determined by ASTM D2765-11, 2006. In another aspect of the composition, the covalently cross-linked hydrogel reaction product exhibits a gel content in the range of 70% to 80% as determined by ASTM D2765-11, 2006. In a further aspect of the composition, the covalently cross-linked hydrogel reaction product exhibits a gel content in the range of 72% to 78% as determined by ASTM D2765-11, 2006.

In yet another aspect of the composition, the reaction mixture comprises the chondroitin 4-sulfate in a range of 0.1% (w/v) to 1.2% (w/v) based on volume of the reaction mixture. In still a further aspect of the composition, the reaction mixture comprises the chondroitin 4-sulfate in a range of 0.2% (w/v) to 0.3% (w/v) based on volume of the reaction mixture. In still another aspect of the composition, the reaction mixture comprises the polyquaternium-10 in a range of 1% (w/v) to 4% (w/v) based on volume of the reaction mixture. In another aspect of the composition, the reaction mixture comprises the polyquaternium-10 in a range of 2% (w/v) to 3% (w/v) based on volume of the reaction mixture. In yet a further aspect of the composition, the reaction mixture comprises the divinylsulfone in a range of 1% (w/w) to 4% (w/w) based on combined weights of the polyquaternium-10 and the chondroitin 4-sulfate in the reaction mixture. In yet another aspect of the composition, the reaction mixture comprises the divinylsulfone in a range of 2% (w/w) to 3% (w/w) based on combined weights of the polyquternium-10 and the chondroitin 4-sulfate in the reaction mixture. In a further aspect of the composition, the reaction mixture comprises the polyquaternium-10 and the chondroitin 4-sulfate in a range of weight ratios of 10:1 to 10:3 based on weights of the polyquaternium-10 and the chondroitin 4-sulfate in the reaction mixture.

In another aspect of the composition, the composition further comprises an isobutylphenylpropionic acid. In a further aspect of the composition, the composition further comprises a Sodium; 2-[2-(2,6-dichloroanilino)phenyl]acetate. In still another aspect of the composition, the covalently cross-linked hydrogel reaction product exhibits a dynamic viscosity at a shear rate of 0.01/s in the range of 3000 Pa to 33000 Pa as determined by ASTM D2084-95, 1994. In yet another aspect of the composition, the covalently cross-linked hydrogel reaction product exhibits a dynamic viscosity at a shear rate of 0.01/s in the range of 10000 to 24000 Pa as determined by ASTM D2084-95, 1994. In still a further aspect of the composition, the covalently cross-linked hydrogel reaction product exhibits a dynamic viscosity at a shear rate of 0.01/s in the range of 15000 to 20000 Pa as determined by ASTM D2084-95, 1994. In yet another aspect of the composition, the reaction mixture comprises 3% (w/v) of polyquaternium-10 based on the volume of the reaction mixture, 0.3% (w/v) of chondroitin 4-sulfate based on the volume of the reaction mixture, and 3% (w/w) of divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.

The present disclosure also encompasses a method of producing the covalently cross-linked hydrogel reaction product of the composition described herein, comprising: combining a first aqueous solution comprising the chondroitin 4-sulfate with a second aqueous solution comprising the polyquaternium-10 to form an aqueous mixture; adding an alkaline solution to the aqueous mixture to form an alkaline aqueous mixture; adding a third aqueous solution comprising the divinylsulfone to the alkaline aqueous mixture to form the reaction mixture; allowing a covalent cross-linking reaction to occur in the reaction mixture to form an intermediate reaction product; neutralizing the intermediate reaction product; washing the intermediate reaction product with a buffered solution; filtering the intermediate reaction product; and, adjusting pH of the intermediate reaction product to form the covalently cross-linked hydrogel reaction product of the composition above. In another aspect of the method, the first aqueous solution comprises the chondroitin 4-sulfate in a range of 0.6% (w/v) to 8.0% (w/v) based on volume of the first aqueous solution. In a further aspect of the method, the first aqueous solution comprises the chondroitin 4-sulfate in a range of 1.2% (w/v) to 4.0% (w/v) based on volume of the first aqueous solution. In yet another aspect of the method, the first aqueous solution comprises the chondroitin 4-sulfate in a range of 1.6% (w/v) to 2.0% (w/v) based on volume of the first aqueous solution. In still a further aspect of the method, the second aqueous solution comprises the polyquaternium-10 in a range of 1.4% (w/v) to 5.7% (w/v) based on volume of the second aqueous solution. In still another aspect of the method, the second aqueous solution comprises the polyquaternium-10 in a range of 3.7% (w/v) to 4.8% (w/v) based on volume of the second aqueous solution. In yet a further aspect of the method, the second aqueous solution comprises the polyquaternium-10 in a range of 4.2% (w/v) to 4.4% (w/v) based on volume of the second aqueous solution. In another aspect of the method, the third aqueous solution comprises the divinylsulfone in a range of 1.2% (w/v) to 4.8% (w/v) based on volume of the third aqueous solution. In a further aspect of the method, the third aqueous solution comprises the divinylsulfone in a range of 2% (w/v) to 4% (w/v) based on volume of the third aqueous solution. In yet another aspect of the method, the third aqueous solution comprises the divinylsulfone in a range of 2.8% (w/v) to 3.2% (w/v) based on the volume of the third aqueous solution. In a further aspect of the method, the reaction mixture comprises 3% (w/v) of polyquaternium-10 based on the volume of the reaction mixture, 0.3% (w/v) of chondroitin 4-sulfate based on the volume of the reaction mixture, and 3% (w/w) of divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.

The present disclosure also encompasses a method of treating a joint of a subject comprising: injecting into the joint the composition described herein. In another aspect of the method of treating a joint the composition as described above is injected into the joint in an amount in the range of 1 ml to 10 ml.

The present disclosure also encompasses a kit for treating joints comprising: the composition as described herein, and a syringe for injecting the composition as described herein into a joint. In another aspect of the kit, the kit further comprises a needle.

The present disclosure also encompasses a composition comprising: a covalently cross-linked hydrogel reaction product of a cross-linking reaction of a reaction mixture comprising a polyquaternium-10, a chondroitin 4-sulfate, and a divinylsulfone, wherein the reaction mixture comprises the chondroitin 4-sulfate in a range of 0.2% (w/v) to 0.3% (w/v) based on volume of the reaction mixture, wherein the reaction mixture comprises the polyquaternium-10 in a range of 2% (w/v) to 3% (w/v) based on volume of the reaction mixture, wherein the reaction mixture comprises the divinylsulfone in a range of 2% (w/w) to 3% (w/w) based on combined weights of the polyquternium-10 and the chondroitin 4-sulfate in the reaction mixture, wherein the chondroitin 4-sulfate, the polyquaternium-10, and the divinylsulfone are covalently cross-linked in the covalently cross-linked hydrogel reaction product, and wherein the covalently cross-linked hydrogel reaction product exhibits a gel content in the range of 72% to 78% as determined by ASTM D2765-11, 2006. In another aspect of the composition, the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises about 46.2% by weight C, about 10.5% by weight H, about 40.3% by weight O, about 1.6% by weight N, and about 1.4% by weight S.

The present disclosure also encompasses a method of producing the covalently cross-linked hydrogel reaction product of the composition as described herein, comprising: combining a first aqueous solution comprising the chondroitin 4-sulfate with a second aqueous solution comprising the polyquaternium-10 to form an aqueous mixture, wherein the first aqueous solution comprises the chondroitin 4-sulfate in a range of 1.6% (w/v) to 2.0% (w/v) based on volume of the first aqueous solution, and wherein the second aqueous solution comprises the polyquaternium-10 in a range of 4.2% (w/v) to 4.4% (w/v) based on volume of the second aqueous solution; adding an alkaline solution to the aqueous mixture to form an alkaline aqueous mixture; adding a third aqueous solution comprising the divinylsulfone to the alkaline aqueous mixture to form the reaction mixture, wherein the third aqueous solution comprises the divinylsulfone in a range of 2.8% (w/v) to 3.2% (w/v) based on the volume of the third aqueous solution; allowing a covalent cross-linking reaction to occur in the reaction mixture to form an intermediate reaction product; neutralizing the intermediate reaction product to form a neutralized intermediate reaction product; washing the neutralized intermediate reaction product with a buffered solution to form a washed neutralized intermediate reaction product; filtering the washed neutralized intermediate reaction product to form a filtered washed neutralized intermediate reaction product; and, adjusting pH of the filtered washed neutralized intermediate reaction product to form the covalently cross-linked hydrogel reaction product of the composition described herein.

The present disclosure also encompasses a method of treating a joint of a subject comprising: injecting into the joint the composition as described herein.

The present disclosure also encompasses a kit for treating joints comprising: the composition as described herein, and a syringe for injecting the composition into a joint.

These and other aspects of the present disclosure are set forth in greater detail below.

### BRIEF DESCRIPTION OF THE DRAWINGS

FIG. 1 shows a polyacrylamide gel electrophoresis of a chondroitin 4-sulfate compared to a reference chondroitin sulfate.

FIG. 2 shows a biocompatible hydrogel composition encompassing aspects of the present disclosure, wherein the biocompatible hydrogel composition is colored with methylene blue after being extruded through a 21-gauge needle and a 27-gauge needle.

FIG. 3 shows a chart illustrating the effect of ionic strength on the stability of two different polyquaternium-10/chondroitin 4-sulfate complexes, wherein a polyquaternium-10/chondroitin 4-sulfate complex with a 1:1.4 M ratio of polyquaternium-10 to chondroitin 4-sulfate is not covalently cross-linked, and another polyquaternium-10/chondroitin 4-sulfate/divinylsulfone complex with a 1:0.5 M ratio of polyquatemium-10 to chondroitin 4-sulfate is covalently cross-linked in a hydrogel.

FIG. 4 shows a chart illustrating turbidity measurements at 400 nm of polyquaternium-10/chondroitin 4-sulfate/divinylsulfone complex covalently cross-linked, illustrated with open circles, and three different polyquaternium-10/chondroitin 4-sulfate complexes that are electrostatically bonded at different charge molar ratios, illustrated with solid squares, solid circles, and open triangles, respectively, wherein the 1st and 3rd cycles comprise constant polyquaternium-10 and increasing chondroitin 4-sulfate, and the 2nd cycle comprises constant chondroitin 4-sulfate and increasing polyquatemium-10.

FIG. 5 shows the radiopacity of prefilled syringes, wherein syringe (1) includes a mixture comprising BaCl₂ mixed with a hydrogel derived from polyquaternium-10, chondroitin 4-sulfate, and divinylsulfone; syringe (2) includes a mixture comprising (15% (w/w)) of MnO₂ mixed with a hydrogel derived from polyquatemium-10, chondroitin 4-sulfate, and divinylsulfone; syringe (3) includes a mixture comprising (20%-40% (w/w)) triyosom mixed with a hydrogel derived from a polyquaternium-10, chondroitin 4-sulfate, and divinylsulfone; and, syringe (4) includes a hydrogel derived from polyquaternium-10, chondroitin 4-sulfate, and divinylsulfone.

FIG. 6 shows a radiograph of a porcine knee implanted with a hydrogel comprising a mixture of a hydrogel derived from polyquaternium-10, chondroitin 4-sulfate, and divinylsulfone and (40% (w/w)) of triyosom and the relative position of the needle and syringe with which the hydrogel was implanted into the knee.

FIG. 7 shows a chart illustrating a thermogravimetric analysis (TGA) thermogram of a hydrogel encompassing aspects of the present disclosure, wherein the results are shown within a temperature range of about 23° C. to about 600° C.

FIG. 8 shows a chart illustrating differential scanning calorimetry (DSC) thermograms of samples C and D showing the three scans (heating/cooling/heating).

FIG. 9 shows a chart illustrating differential scanning calorimetry (DSC) thermogram of sample D, which encompasses aspects of the present disclosure.

### DETAILED DESCRIPTION

The present disclosure is directed to biocompatible hydrogel compositions comprising covalently cross-linked hydrogel reaction products of reaction mixtures comprising a cross-linking agent, divinylsulfone, a natural polymer of animal origin, chondroitin 4-sulfate, and a natural polymer of vegetal origin modified by the incorporation of quaternary ammonium groups, polyquaternium-10, wherein the chondroitin 4-sulfate is covalently cross-linked with the polyquaternium-10 and the divinylsulfone, thereby forming an amorphous complex constituting a hydrogel. The covalently cross-linked hydrogel reaction products and compositions comprising the products can be colorless and/or transparent. The compositions of the present disclosure can comprise a polyelectrolyte complex interpenetrating hydrogel, made of two independent cross-linked naturally derived polymers, chondroitin sulfate and a modified cellulose derivative that forms a three-dimensional structure. The present disclosure also is directed to methods of making the biocompatible hydrogel compositions, methods of using the biocompatible hydrogel compositions, including methods of treatment of a subject, and kits comprising the compositions.

As used herein, the singular forms of “a,” “an,” and “the” encompasses the plural form thereof unless otherwise indicated. As used herein, the phrase “at least one” includes all numbers of one and greater. The ranges used herein include all values that would fall within the stated range, including values falling intermediate of whole values, and are inclusive of the minimum and maximum values. As used herein, the term “and/or” refers to one or all of the listed elements or a combination of any two or more of the listed elements. As used herein, the values described as “% (w/w)” are calculated on the weight of the specified composition and the weight of the specified component, or the weight of the specified component and the combined weights of other specified components. As used herein, the values described as “% (w/v)” are calculated on the weight of the specified component and the volume of the specified composition containing the specified component. As used herein, “C” means carbon, “0” means oxygen, “H” means hydrogen, “N” means nitrogen, and “S” means sulfur.

As used herein, chondroitin 4-sulfate is a sulfated glycosaminoglycan composed of a chain of alternating sugars (N-acetylgalactosamine and glucuronic acid) with a molecular weight that depends on its source. Chondroitin sulfate is otherwise known as (2S,3S,4S,5R,6R)-6-[(2R,3R,4R,5R,6R)-3-acetamido-2,5-dihydroxy-6-sulfooxyoxan-4-yl]oxy-3,4,5-trihydroxyoxane-2-carboxylic acid. Chondroitin sulfate can have a molecular formula of C₁₃H₂NO₁₅S. The chondroitin 4-sulfate chain is formed by about 100 individual sugars, which can be sulfated in variable amounts and in different positions. Chondroitin 4-sulfate is a necessary structural component of cartilage and provides the resistance to compression. The chondroitin 4-sulfate loss in cartilage is one of the main causes of osteoarthritis disease. The term chondroitin 4-sulfate, as used herein, means a chondroitin sulfate in sodium salt form with a sulfate group at the 4-position of the molecular structure (C-4S) that can have a structure as shown below.

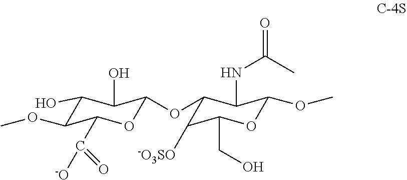

As used herein, polyquatemium-10, sometimes referred to as PQ-10, is a hydroxyethyl cellulose substituted with quaternary amine groups, and is otherwise known as [3-[2[(2R,3R,4S,5S,6R)-2,4-dihydroxy-5-[(3R,4R,5R,6R)-3-hydroxy-4,5-bis(2-hydroxyethoxy)-6-(hydroxymethyl)oxan-2-yl]oxy-6-(hydroxymethoxymethyl)oxan-3-yl]oxyethoxy]-2-hydroxypropyl]-trimethylazanium; chloride. Polyquaternium-10 can have a molecular formula of C₂₅H₅₀CINO₁₆. Polysaccharides containing quaternary ammonium groups exhibit solubility in aqueous media, biocompatibility, and mucoadhesion properties. Polyquaternium-10 can have the following molecular structure:

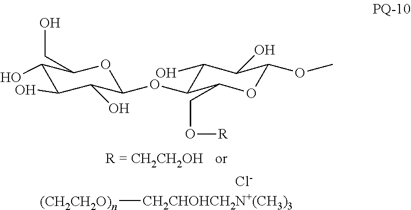

As used herein, divinylsulfone to a cross-linking agent that is sometimes referred to as 1-ethenylsulfonylethene and that has a molecular formula of C₄H₆O₂S. As used herein, “covalent cross-linking reaction” means a cross-linking reaction that results in a chemical covalent bond between the chains of different molecules or polymers. As used herein, “gel content” means the ratio of the mass of the insoluble residue divided by the initial mass of the test sample, resulting from the Soxhlet extraction method described in ASTM D2765-11.

As used herein, the term “hydrogel” refers to a three-dimensional network of molecules that are covalently bound to each other. As used herein, the terms “treatment” and “treating” refers to any treatment of any one or more conditions, diseases, disorders, and/or injuries in a subject, and can include: inhibiting the condition, disease, disorder, and/or injury, (2) relieving the condition, disease, disorder, and/or injury, or any one or more symptoms thereof, and/or (3) ameliorating the disease, disorder, and/or injury, or any one or more symptoms thereof. As used herein, the term “subject” refers to any animal, such as a mammal, such as a human to which a treatment can be administered. As used herein, the term “pH” and “pH value” refer to the logarithm of the reciprocal of the hydrogen activity in a solution expressed in decimal form. As used herein, the term “biocompatible” refers to materials that do not induce a substantial detrimental response in vivo.

The covalently cross-linked hydrogel reaction products of the present disclosure can be formed from a reaction mixture comprising an aqueous solution, a polyquaternium-10, a chondroitin 4-sulfate, and a divinylsulfone. The reaction mixtures of the present disclosure can comprise about 1% (w/v) to about 4% (w/v) of a polyquaternium-10 based on volume of the reaction mixture. In one aspect, the upper limit of the range of the polyquatermiun-10 in the reaction mixture can be about 3% (w/v) or 4% (w/v) based on volume of the reaction mixture. In another aspect, the upper limit of the range of the polyquatermiun-10 in the reaction mixture can be about 3.1% (w/v), 3.2% (w/v), 3.3% (w/v), 3.4% (w/v), 3.5% (w/v), 3.6% (w/v), 3.7% (w/v), 3.8% (w/v), or 3.9% (w/v) based on volume of the reaction mixture. In still another aspect, the lower limit of the range of the polyquaternium-10 in the reaction mixture can be about 1% (w/v), 2% (w/v), or 3% (w/v) based on volume of the reaction mixture. In yet another aspect, the lower limit of the range of the polyquaternium-10 in the reaction mixture can be about 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), 2.1% (w/v), 2.2% (w/v), 2.3% (w/v), 2.4% (w/v), 2.5% (w/v), 2.6% (w/v), 2.7% (w/v), 2.8% (w/v), or 2.9% (w/v) based on volume of the reaction mixture. In further aspect, the reaction mixture can comprise be about 3% (w/v) of polyquaternium-10 based on volume of the reaction mixture.

The reaction mixture can comprise about 0.1% (w/v) to about 1.2% (w/v) of a chondroitin 4-sulfate based on the volume of the reaction mixture. In one aspect, the lower limit of the range of the chondroitin 4-sulfate in the reaction mixture can be about 0.1% (w/v), 0.2% (w/v), or 0.3% (w/v) based on volume of the reaction mixture. In another aspect, the upper limit of the range of the chondroitin 4-sulfate in the reaction mixture can be about 0.4% (w/v), 0.5% (w/v), 0.6% (w/v), 0.7% (w/v), 0.8% (w/v), 0.9% (w/v), 1.0% (w/v), 1.1% (w/v), or 1.2% (w/v) based on volume of the reaction mixture. In a further aspect, the reaction mixture can comprise about 0.3% (w/v) of a chondroitin 4-sulfate based on volume of the reaction mixture.

The reaction mixture can comprise about 1% (w/w) to about 4% (w/w) of a divinylsulfone based on combined weights of the polyquatemium-10 and the chondroitin 4-sulfate in the reaction mixture. In one aspect, the lower limit of the range of the divinylsulfone in the reaction mixture can be about 1% (w/w), 2% (w/w), or 3% (w/w) based on combined weights of the polyquatemium-10 and the chondroitin 4-sulfate in the reaction mixture. In another aspect, the lower limit of the range of the divinylsulfone in the reaction mixture can be about 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.1% (w/w), 2.2% (w/w), 2.3% (w/w), 2.4% (w/w), 2.5% (w/w), 2.6% (w/w), 2.7% (w/w), 2.8% (w/w), or 2.9% (w/w) based on combined weights of the polyquatemium-10 and the chondroitin 4-sulfate in the reaction mixture. In another aspect, the upper limit of the range of the divinylsulfone in the reaction mixture can be about 3.1% (w/w), 3.2% (w/w), 3.3% (w/w), 3.4% (w/w), 3.5% (w/w), 3.6% (w/w), 3.7% (w/w), 3.8% (w/w), or 3.9% (w/w) based on combined weights of the polyquaternium-10 and the chondroitin 4-sulfate in the reaction mixture. In a further aspect, the reaction mixture can comprise about 3% (w/w) of a divinylsulfone based on combined weights of the polyquatemium-10 and the chondroitin 4-sulfate in the reaction mixture.

The gel content exhibited by the covalently cross-linked hydrogel reaction product of the present disclosure can be in the range of about 65% to about 85%, as determined by ASTM D2765-11, 2006. In one aspect, the upper limit of the range of the gel content exhibited by the covalently cross-linked hydrogel reaction product can be about 74%, 75%, 76%, 77%, 78%, 79%, 80%, 81%, 82%, 83%, 84%, or 85%. In another aspect, the lower limit of the range of the gel content exhibited by the covalently cross-linked hydrogel reaction product can be about 65%, 66%, 67%, 68%, 69%, 70%, 71%, 72%, or 73%. In a further aspect, the covalently cross-linked hydrogel reaction product can exhibit a gel content of about 74%.

The pH value of the covalently cross-linked hydrogel reaction product can be in the range of about 5.5 to about 7.5. In another aspect, the pH value of the covalently cross-linked hydrogel reaction product can be in the range of about 6.0 to about 7.0. In yet another aspect, the pH value of the covalently cross-linked hydrogel reaction product can be in the range of about 6.0 to about 6.5. In still a further aspect, the pH value of the covalently cross-linked reaction product can be about 6.2.

The covalently cross-linked hydrogel reaction product can exhibit a dynamic viscosity at 0.01 s⁻¹ in a range of about 3300 Pa to about 33000 Pa as determined by ASTM D2084-95, 1994 for determination of rheometric characteristics using a Physica Rheometer Rheoplus/32 available from Anton Paar USA, Inc. of Ashland, Va., USA. In another aspect, the covalently cross-linked hydrogel reaction product can exhibit a dynamic viscosity at 0.01 s⁻¹ in a range of about 10000 Pa to about 23000 Pa. In yet another aspect, the covalently cross-linked hydrogel reaction product can exhibit a dynamic viscosity at 0.01 s⁻¹ in a range of about 15000 Pa to about 20000 Pa. In one aspect, the upper limit of the range of the dynamic viscosity at 0.01 s⁻¹ can be about 20000 Pa, 21000 Pa, 22000 Pa, 23000 Pa, 24000, Pa, 25000 Pa, 26000 Pa, 27000 Pa, 28000 Pa, 29000 Pa, 30000 Pa, 31000 Pa, 32000 Pa, or 33000 Pa. In another aspect, the lower limit of the range of the dynamic viscosity at 0.01 s⁻¹ can be about 3000 Pa, 4000 Pa, 5000 Pa, 6000 Pa, 7000 Pa, 8000 Pa, 9000 Pa, 10000 Pa, 11000 Pa, 12000 Pa, 13000 Pa, 14000 Pa, or 15000 Pa.

The present disclosure encompasses methods of making covalently cross-linked hydrogel reaction products that comprise preparation of a mixture of polysaccharide with glycosaminoglycan in such a proportion as to form a hydrogel of maximum consistency given by electrostatic and hydrophobic interactions. In one aspect, the mixture can comprise about 2%-4% (w/v) of the polycation and between 0.2%-0.4% (w/v) of glycosaminoglycan based on the volume of the reaction mixture. In another aspect, the mixture can comprise about 10% by weight with respect to the polycation. To the reaction mixture can be added a cross-linking agent, divinylsulfone, in different weight percentages based on the initial combined weights of the polymers. In one aspect, the divinylsulfone can be added in a range of 1%-4% (w/w) based on the combined weight of the polymers polyquaternium-10 and chondroitin 4-sulfate.

The present disclosure encompasses a method of making compositions comprising covalently cross-linked hydrogel reaction products comprising the following steps: (a) combining of first aqueous solution comprising chondroitin 4-sulfate with a second aqueous solution comprising polyquaternium-10 to form an aqueous mixture; (b) adding an alkaline solution to the aqueous mixture to form an alkaline aqueous mixture; (c) adding divinylsulfone in a third aqueous solution to the alkaline aqueous mixture to form a reaction mixture; (d) allowing a covalent cross-linking reaction to occur in the reaction mixture during a pre-determined time to obtain an intermediate covalently cross-linked hydrogel reaction product; (e) neutralizing the intermediate covalently cross-linked hydrogel reaction product to form a neutralized covalently cross-linked hydrogel reaction product; (f) washing the neutralized covalently cross-linked hydrogel reaction product with a glycine-phosphate buffer solution comprising about 0.1% (w/v) of glycine to block the unreacted divinylsulfone; (g) filtering the mixture of neutralized covalently cross-linked hydrogel reaction product and the glycine-phosphate buffer solution to remove unreacted divinylsulfone; (h) repeating steps (f) and (g) in multiple cycles to form a washed, filtered, and neutralized covalently cross-linked hydrogel reaction product; and, (i) adjusting the pH of the washed, filtered, and neutralized covalently cross-linked hydrogel reaction product to a pre-determined range of pH appropriate for the intended use to form a covalently cross-linked hydrogel reaction product.

In one aspect, step (d) of mixing the reaction mixture to allow for the cross-linking reaction can be carried out with the reaction mixture having a temperature range of about 0° C. to about 25° C. In another aspect, intended use to form a covalently cross-linked hydrogel reaction product. In one aspect, step (d) of mixing the reaction mixture to allow for the cross-linking reaction can be carried out with the reaction mixture having a temperature in a temperature range of about 20° C. to about 25° C. In a further aspect, the lower limit of the temperature range of the reaction mixture during step (d) can be about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., or 24° C. In a further aspect, the upper limit of the temperature range of the reaction mixture during step (d) can be about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. In still another aspect, the temperature of the reaction mixture during step (d) can be about 23° C. In a further aspect, the temperature of the reaction mixture during step (d) can be about 4° C. The method of making compositions comprising covalently cross-linked hydrogel reaction products comprising can be carried out at atmospheric pressure. In a further aspect, the pre-determined time period for step (d) is at least 12 hours. In yet another aspect, the pre-determined time period for step (d) is within a range of about 10 hours to about 14 hours. In another aspect, the reaction mixture in can be stirred in step (d).

The present disclosure encompasses compositions comprising covalently cross-linked hydrogel reaction products and methods of making the reaction products from cationic polysaccharides covalently cross-linked with anionic glycosaminoglycan in proportions that can, in some embodiments, impart higher consistency to the hydrogel reaction product than would otherwise exhibited by a gel formed by non-covalent electrostatic and/or hydrophobic interactions.

Some of the compositions comprising covalently cross-linked hydrogel reaction products encompassed by the present disclosure are made by a method of carrying out a covalent cross-linking reaction in a reaction mixture comprising a polyquaternium-10 and a chondroitin 4-sulfate in weight ratio of about 10:1. With such a ratio of polyquaternium-10 to chondroitin 4-sulfate, the resulting covalently cross-linked hydrogel reaction products can, in some embodiments, exhibit a level of mucoadhesivity that can make the compositions comprising the covalently cross-linked hydrogel reaction product suitable for certain biomedical applications. Without being bound to a particular theory, the polyquaternium-10-derived portions of the covalently cross-linked hydrogel reaction product can exhibit a positive charge that is able to interact with mucosal layers that tend to be negatively charged.

The chondroitin 4-sulfate, with molecular weight of 14.4-31 kg/mol, with a purity not less than 97%, is solubilized in distilled water at concentration of 0.1%-0.4% (w/v) to form a first aqueous solution. The commercial polyquaternium-10 in powder form, exhibiting a viscosity 700-2100 CP at 1% (w/v) is solubilized in distilled water at concentration of 2%-4% (w/v) to form a second aqueous solution. The first and second aqueous solutions are mixed together under vigorous stirring to form an aqueous mixture. Then an alkaline solution comprising sodium hydroxide (NaOH) is added to the aqueous mixture, resulting in an alkaline aqueous mixture having a concentration of 50 mM of NaOH. A third aqueous solution comprising a divinylsulfone dissolved in distilled water is added to the alkaline aqueous mixture in proportions ranging from 1% (w/w) to 4% (w/w) based on the combined weights of the polyquaternium-10 and the chondroitin 4-sulfate contained in aqueous mixture, thereby forming a reaction mixture. A covalent cross-linking reaction is allowed to occur in the reaction mixture for a pre-determined time period. The reaction mixture can be stirred during the reaction.

The covalent cross-linking reaction results in the formation intermediate reaction product comprising a covalently cross-linked hydrogel reaction product that tends to prevent the stirring mechanism from moving due to the viscosity of the covalently cross-linked hydrogel reaction product. The covalently cross-linked hydrogel reaction product can be clear and/or translucent and/or transparent. The covalently cross-linked hydrogel reaction product so formed is allowed to stand for a pre-determined time period, which can be at least 12 hours, to allow the covalent cross-linking reaction to occur as fully as possible. The pre-determined time period can be in a range of about 10 hours to about 14 hours. After this pre-determined time period, the intermediate reaction product is neutralized by the addition of a 50 mM hydrogen chloride (HCl) solution of a volume equal to that of the reaction mixture, and is allowed to neutralize for about one hour. The neutralized intermediate reaction product is extracted and successive washing procedures of the hydrogel reaction product are carried out with phosphate buffer containing 0.1% (w/v) of glycine, repeating cycles of washing and removal of the supernatant by filtering can be carried out about two times per day for at least one week. Accordingly, the washing and filtering steps can be repeated in a range of thirty to fifty times. The pH of the neutralized, washed and filtered intermediate reaction product can be adjusted to within a range of about 6.5 to about 7.0. The temperature of the intermediate reaction product comprising a covalently cross-linked hydrogel reaction product can be maintained in a temperature range of about 4° C. to about 8° C. until the intermediate reaction product can be sterilized. The covalently cross-linked hydrogel reaction product can be sterilized by exposure to steam during an autoclave cycle occurring in a time period of about 15 minutes to about 20 minutes in a temperature range of about 120° C. to about 123° C.

The covalently cross-linked hydrogel reaction product that is produced from the method of set forth herein can be lyophilized to remove substantially all the water therefrom and subsequently rehydrated with the addition of water thereto to a desired concentration. In addition, the lyophilized and powdered covalently cross-linked hydrogel reaction product can be hydrated in other suitable solvents, including aqueous buffered solutions that are combined with pharmacologically active molecules, such as anti-inflammatory agents, anticoagulants, etc.

The final elemental composition of the purified covalently cross-linked hydrogel reaction product, produced from a reaction mixture comprising 3% (w/v) of polyquaternium-10, 0.3% (w/v) of chondroitin 4-sulfate, and 3% (w/w) of divinylsulfone based on the combined weights of the polyquaternium-10 and the chondroitin 4-sulfate in the reaction mixture, was about 46.2% C, about 10.5% H, about 40.3% O, about 1.6% N, and about 1.4% S, as determined using an Elemental Analyzer CHNSO Series II and EA 2400 Data Manager both available from PerkinElmer of Boston, Mass., United States, and an Autoanalyzer SmartChem 200 from Unity Scientific of Milford, Mass., United States.

The present disclosure encompasses methods of treatment comprising applying the compositions comprising biocompatible hydrogels disclosed herein. In one aspect, a method of treating a joint of a subject comprises applying to a subject any one or more of the compositions disclosed herein. A method of treating a joint can comprise the step of injecting into the joint a composition comprising a covalently cross-linked hydrogel reaction product produced from a reaction mixture comprising polyquaternium-10, chondroitin 4-sulfate, and divinylsulfone. The present disclosure also encompasses kits comprising one or more of the compositions disclosed herein and a delivery device that can be used to administer the composition to a subject. The delivery device can include a syringe, either singly or in combination with a needle, or other suitable device for administering the compositions disclosed herein to a subject.

### EXAMPLES

### General Procedure for the Determination of Gel Content

For determination of the gel content, tests were conducted pursuant to the “American Society for Testing and Materials, Standard Test Methods for Determination of Gel Content and Swell Ratio of Cross-linked Ethylene Plastics, ASTM D2765-11, 2006”.

The procedure carried out included the following: each sample was washed with distilled water to separate the non-cross-linked fraction. Then, the remaining cross-linked and, hence, insoluble complex (“gel”) was dried at 80° C. for 3 days, followed by the determination of its net weight (M₂). The ratio of the mass of the insoluble residue divided by the initial mass of all components of the complex (M₁) yields the method's measure and “gel content”:

$$Gel content ( \% ) = ( \frac{{M}_{2}}{{M}_{1}} ) 100 , {M}_{2} \leq {M}_{1} \qquad Equation 1$$

Where M₁ is the initial mass of all components of the complex, and M₂ is the mass of the same complex after the drying process.

### General Procedure for Dynamic Viscosity and Storage Module Measurement

For determination of dynamic viscosity and storage module, tests were conducted pursuant to the “American Society for Testing and Materials, Standard Test Methods for Determination of Rubber Property-Vulcanization Using Oscillating Disk Cure Meter, ASTM D2084-95, 1994”.

The procedure carried out included the following: a 5 cm cone-plate measuring geometry was used. Oscillatory shear responses (G′ or storage modulus, and G″ or loss modulus) were determined at 0.1 Pa over the frequency range of 0.1-100 rad s-1. Samples were evaluated in triplicate at 25° C. The test conditions were within the linearity range of the viscoelastic properties.

### Example 1: Determination of Chondroitin 4-Sulfate Molecular Weight (MW) by the SDS-PAGE Electrophoresis Technique Using a Polyacrylamide Gel

Polyacrylamide gel electrophoresis was performed using the method described in “Polyacrylamide-gel electrophoresis and Alcian Blue staining of sulphated glycosaminoglycan oligosaccharides” by Mary K. Cowman, Mary F. Slahetka, Daniel M. Hittner, Jiyun Kim, Michael Forino and GeihanGadelrab. Biochem. J. (1984) (221, 707-716). An acrylamide solution, containing 30% acrylamide and 0.8% NN′-methylene bisacrylamide, was used and protected from light and stored at 4° C. Concentrated buffers, containing 1.5 M tris(hydroxymethyl)aminomethane/hydrogen chloride at a pH 8.6, also preserved at 4° C., were used for the gel separator. A solution of 0.5 M tris(hydroxymethyl)aminomethane/hydrogen chloride at a pH 6.8 was used for the concentrator gel. For the polymerization, solutions of 50 μl and 70 μl of 10% (w/v) persulfate solution (prepared at the time for the separator gel) were used for the concentrator gel. In addition, a 10 μl solution of concentrated NNN′N′-tetramethylenediamine (TEMED) and 100 μl of 10% (w/v) sodium dodecyl sulfate (SDS) were added for a 10 ml amount of gel solution. The gels, each with an area of 7 cm long by 10 cm wide, were prepared between two glass plates spaced 0.75 cm apart. The sowing areas, each 1.5 cm deep and 0.5 cm wide, were formed by a 10-tooth PERSPEX® acrylic comb and were allowed to polymerize overnight at room temperature before use.

To run the electrophoresis test, samples containing 10 μg and 20 μg of chondroitin 4-sulfate (from a concentrated solution) were mixed with 1:4 volumes of buffer composed of glycerol, 0.5 M tris(hydroxymethyl)aminomethane/hydrogen chloride buffer at a pH 6.8, 8% (w/v) sodium dodecyl sulfate, and 0.4% (w/v) bromophenol blue (4× sample buffer). A sample of low molecular weight proteins was used as the marker, and was put in a separate lane, and a mixed sample of protein and chondroitin 4-sulfate was put in another line. The same process was carried out with a reference sample of chondroitin sulfate obtained from Biotech Lab S.R.L. of Buenos Aires, Argentina.

The buffer used to run the gel was a 1:4 dilution of 0.1 M tris(hydroxymethyl)aminomethane and 0.77 M Glycine at a pH 8.3, to which 0.1% (w/v) sodium dodecyl sulfate was then added. The gel was connected to a potential source at 65 V for 1 hour and then at 110 V for 2 hours. The gels were then stained with a 0.5% solution of alcian Blue in 2% acetic acid for 45 minutes, and then bleached in 2% acetic acid for 15 minutes.

A second staining with COOMASSIE® Brilliant Blue Solution in 50% methanol, 40% water, and 10% acetic acid was then made to color the molecular weight marker, and finally the latter was bleached with a solution of 10% acetic acid and 10% methanol.

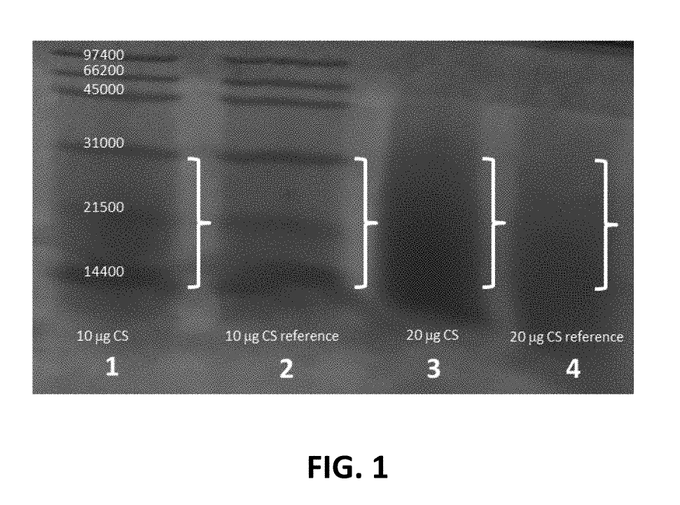

FIG. 1 shows the profiles obtained for two lanes with the sample plus the marker and two lanes with the sample at another concentration. The first line of FIG. 1 shows a BIO-RAD® low molecular weight protein marker and 10 μg of a chondroitin 4-sulfate. The second line of FIG. 1 shows a BIO-RAD® low molecular weight protein marker and 10 μg of a reference chondroitin sulfate. The third line of FIG. 1 shows 20 μg of a chondroitin 4-sulfate, and the fourth line of FIG. 1 shows 20 μg of a reference chondroitin sulfate. In general, it is observed that the bands are wide, evidencing dispersity in the chondroitin 4-sulfate that was analyzed. Based on these results, the apparent molecular weight of the chondroitin 4-sulfate tested is between 14.4 and 31.0 kg/mol with respect to the protein marker used as a standard.

### Example 2: Determination of Polyquaternium-10 Viscosity

A polyquaternium-10 solution (2%) in distilled water was prepared, from which the corresponding dilutions were made in order to obtain final concentrations of: 0.5% (w/v); 1% (w/v), and 1.5% (w/v). For these solutions, the viscosity was determined using a Brookfield viscometer, cone-plate geometry (CP52) at 20° C. and at different rotational speeds.

TABLE 1

| | Speed | Force | Viscosity |
|---|---|---|---|
| Sample | (rpm) | (%) | (cps) |
| polyquatemium-10 (0.5%) | 10 | 13.5 | 125.5 |
| | 20 | 22.2 | 102.8 |
| | 30 | 28.8 | 89.3 |
| | 40 | 34.5 | 80.2 |
| | 60 | 44.3 | 68.7 |
| | 80 | 52.7 | 61.3 |
| | 100 | 60.2 | 56.1 |
| | 120 | 66.9 | 51.8 |
| | 160 | 78.6 | 45.7 |
| polyquatemium-10 (1%) | 0.5 | 11.1 | 2083 |
| | 1 | 20.3 | 1888 |
| | 2 | 32.4 | 1516 |
| | 5 | 57.4 | 1068 |
| | 10 | 83.9 | 778 |
| | 20 | — | 736* |
| polyquatemium-10 (1.5%) | 0.1 | 13 | 12600 |
| | 0.5 | 46 | 8667 |
| | 1.0 | 73 | 6845 |
| | 20 | — | 5967* |
| | 20 | — | 3916* |

*Viscosity value extrapolated to 20 RPM by adjusting the curve viscosity vs. speed.

Based on these test results, the polyquaternium-10 samples at a concentration of 1% (w/v) and 0.5 rpm have a viscosity of 2083 cp

### Example 3: Preparation of a Covalently Cross-Linked Hydrogel Reaction Product of a Cross-Linking Reaction of a Polyquaternium-10, a Chondroitin 4-Sulfate, and a Divinylsulfone, Wherein the Covalently Cross-Linked Hydrogel Reaction Product Comprises 3% (w/v) of the Polyquaternium-10, 0.3% (w/v) of the Chondroitin 4-Sulfate, and 3% (w/w) (Based on the Total Weight of the Polyquaternium-10 and the Chondroitin 4-Sulfate) of the Divinylsulfone

A sample of 0.3 grams of polyquaternium-10 was dissolved in 7 ml of distilled water, and a sample of 0.03 grams of chondroitin 4-sulfate was dissolved in 1.5 ml of distilled water. Both solutions were mixed under vigorous stirring at room temperature for 5 minutes to allow electrostatic interaction between the two polymers. Then 1 ml of 0.5 M NaOH solution was added to reach a final concentration of 50 mM NaOH and the appropriate pH. An amount of 330 μl of a 3% (w/v) divinylsulfone dilution in distilled water was added to the mixture, and then 170 μl of distilled water was added to reach a final volume of 10 ml, thereby obtaining 3% (w/w) of the divinylsulfone cross-linking agent in relation to the initial mass of the two combined polymers. The combined reaction mixture was stirred vigorously for about one minute, after which the formation of a transparent three-dimensional hydrogel reaction product could be seen. The reaction mixture was then allowed to continue to react for at least 12 hours at 4° C. The formed covalently cross-linked hydrogel reaction product was transferred to 10 ml of a 50 mM hydrogen chloride solution, and allowed to stand for one hour. After this time, the covalently cross-linked hydrogel reaction product was washed several times with a phosphate buffer solution containing 0.1% (w/v) of glycine to remove the unreacted divinylsulfone and to adjust the pH of the covalently cross-linked hydrogel reaction product to a pH in the range of 6.5 to 7, which is similar to the pH of physiological conditions in which the covalently cross-linked hydrogel reaction product could be used. Between the washing steps, the submerged covalently cross-linked hydrogel reaction product was allowed to stand in the wash solution at 4° C. Successive washes of the covalently cross-linked hydrogel reaction product were carried out during the course of one week.

Table 2 shows the different ratios of polyquaternium-10 and chondroitin 4-sulfate used to produce the covalently cross-linked hydrogel reaction products.

TABLE 2
Example 3: SAMPLE COMBINATIONS

| | | Chondroitin | | |
|---|---|---|---|---|
| | Polyquaternium-10 | 4-sulfate | Weight | Divinylsulfone |
| Test | % (w/v) | % (w/v) | ratio^{a} | % (w/w)^{b} |
| 1-1 | 1 | 0.1 | 10:1 | 1 |
| 1-2 | 1 | 0.1 | 10:1 | 2 |
| 1-3 | 1 | 0.1 | 10:1 | 3 |
| 1-4 | 1 | 0.1 | 10:1 | 4 |
| 1-5 | 1 | 0.1 | 10:1 | 5 |
| 2-1 | 2 | 0.2 | 10:1 | 1 |
| 2-2 | 2 | 0.2 | 10:1 | 2 |
| 2-3 | 2 | 0.2 | 10:1 | 3 |
| 3-1 | 3 | 0.3 | 10:1 | 1 |
| 3-2 | 3 | 0.3 | 10:1 | 2 |
| 3-3 | 3 | 0.3 | 10:1 | 3 |
| 3-4 | 3 | 0.3 | 10:1 | 4 |
| 4-3 | 4 | 0.4 | 10:1 | 3 |
| 4-4 | 4 | 0.4 | 10:1 | 4 |
| 4-5 | 4 | 0.8 | 10:2 | 4 |
| 4-6 | 4 | 1.2 | 10:3 | 4 |

TABLE notes:
^{a}Weight ratio between polyquatemium-10 and chondroitin 4-sulfate.
^{b}Weight percentage of the di vinyl sulfone cross-linking agent in respect to the total weight of the two polymers.

The covalently cross-linked hydrogel reaction product with a concentration of polyquaternium-10 at 3% (w/v), chondroitin 4-sulfate at 0.3% (w/v), and divinylsulfone at 3% (w/w) (based on the combined weight of the polyquaternium-10 and the chondroitin 4-sulfate) was selected to test for biocompatibility and intra-articular implantation. This covalently cross-linked hydrogel reaction product was selected based on its rheological properties.

### Example 4: Extrudability Through Needles of 21 Gauge and 27 Gauge of a Selected Covalently Cross-Linked Hydrogel Reaction Product Produced in Example 3

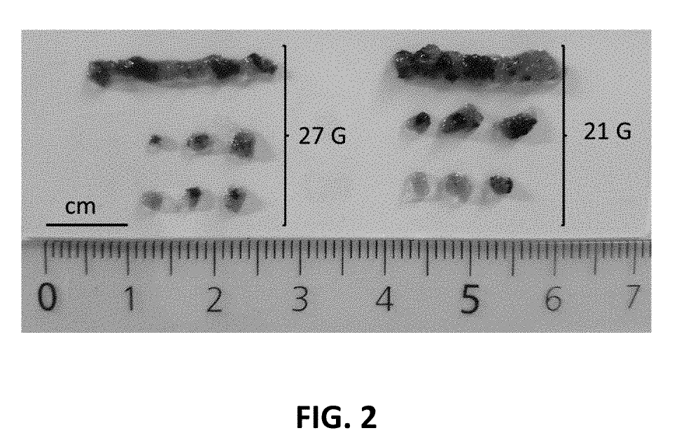

FIG. 2 shows a photograph of the selected covalently cross-linked hydrogel reaction product stained with methylene blue (used for better visualization), after being extruded through needles of different diameters, one needle being 21 gauge and the other being 27 gauge. The covalently cross-linked hydrogel reaction product was manually extruded through both needles to mimic possible use in intra-articular injection. It was observed that the covalently cross-linked hydrogel reaction product had a particle size of about 1-2 mm when extruded through the 27-gauge needle, and a particle size of about 3-4 mm when extruded through the 21-gauge needle.

### Example 5: Rheology Tests of the Covalently Cross-Linked Hydrogel Reaction Products Produced in Example 3

For determination of dynamic viscosity and the storage modulus, tests were conducted pursuant to the “American Society for Testing and Materials, Standard Test Methods for Determination of Rubber Property-Vulcanization Using Oscillating Disk Cure Meter, ASTM D2084-95, 1994” in accordance with the above described “General procedure for dynamic viscosity and the storage modulus measurement”.

In this test, the dynamic viscosity and the storage modulus were determined for different covalently cross-linked hydrogel reaction products of cross-linking reactions of a polyquaternium-10, a chondroitin 4-sulfate, and a divinylsulfone cross-linking agent, using the procedures and combinations described in Example 3. These same properties were also measured for control samples, which consisted of the same combinations of the polymers polyquaternium-10 and a chondroitin 4-sulfate without the presence of the divinylsulfone cross-linking agent. Comparisons with a control samples were performed in order to observe the change in the rheological properties that occurred when a covalent cross-linking reaction was carried out. Samples were measured before being washed with phosphate buffer saline solution.

Table 3 shows that the covalent cross-linking reaction of the cationic polymer with the glycosaminoglycan provides a substantial increase in both the dynamic viscosity and the storage module of the resulting covalently cross-linked hydrogel reaction products as compared to the combination of the same polymers without the covalent cross-linking reaction.

TABLE 3
Example 5: DYNAMIC VISCOSITY AND ELASTIC MODULE MEASUREMENT

| | | | | | Storage Module | | |
|---|---|---|---|---|---|---|---|
| | Dynamic viscosity (Pa) | | | | (G′, Pa) at different | | |
| | at different shear rates (s⁻¹) | | | | frequencies (Hz) | | |
| Test | 0.01 | 0.46 | 10 | 100 | 0.1 | 4 | 40 |
| polyquaternium-10-1%^{a} | 18.6 | 8.9 | 2.2 | 0.54 | 0.52 | 9.8 | 33.4 |
| polyquaternium-10-1%- | 133 | 17.0 | 2.6 | 0.59 | 4.7 | 19.1 | 41.2 |
| chondroitin 4-sulfate- | | | | | | | |
| 0.1%^{a} | | | | | | | |
| 1-1 | 441 | 54.0 | 4.8 | 0.72 | 6.8 | 27.2 | 44.3 |
| 1-2 | 661 | 67.0 | 5.6 | 0.61 | 14.5 | 44.4 | 63.6 |
| 1-3 | 3436 | 84.0 | 6.4 | 0.68 | 14.1 | 34.2 | 49.9 |
| polyquaternium-10-2%^{a} | 89 | 30.0 | 6.0 | 1.2 | 4.1 | 37.4 | 106 |
| polyquaternium-10-2%- | 665 | 76.0 | 9.7 | 1.7 | 31.8 | 107 | 192 |
| chondroitin 4-sulfate- | | | | | | | |
| 0.2%^{a} | | | | | | | |
| 2-1 | 2334 | 236 | 12.0 | 0.62 | 76.8 | 279 | 387 |
| 2-2 | 75586 | 95.7 | 12.4 | 0.51 | 104 | 532 | 789 |
| polyquaternium-10-3%^{a} | 509 | 105 | 16.6 | 2.8 | 21.9 | 122 | 278 |
| polyquaternium-10-3%- | 1793 | 157 | 18.1 | 2.9 | 68.1 | 239 | 419 |
| chondroitin 4-sulfate- | | | | | | | |
| 0.3%^{a} | | | | | | | |
| 3-1 | 67640 | 590 | 23.0 | 0.80 | 119 | 419 | 655 |
| 3-2 | 40389 | 711 | 8.0 | 0.48 | 158 | 343 | 467 |
| 3-3^{b} | — | — | — | — | — | — | — |
| 3-4 | 26508 | 934 | 36.0 | 1.3 | — | 396 | 402 |
| 4-3 | 54922 | 2214 | 84.0 | 2.3 | — | 790 | 813 |
| 4-4^{b} | — | — | — | — | — | — | — |

TABLE notes:
^{a}Control sample without cross-linking. Percentage given in (w/v) units.
^{b}Too rigid hydrogel. Viscosity and module exceeded the measurement limit of the instrument.

### Example 6: Stability of Covalently Cross-Linked Hydrogel Reaction Products of the Cross-Linking Reaction of Polyquaternium-10, Chondroitin 4-Sulfate, and Divinylsulfone in Solutions with Ionic Strength

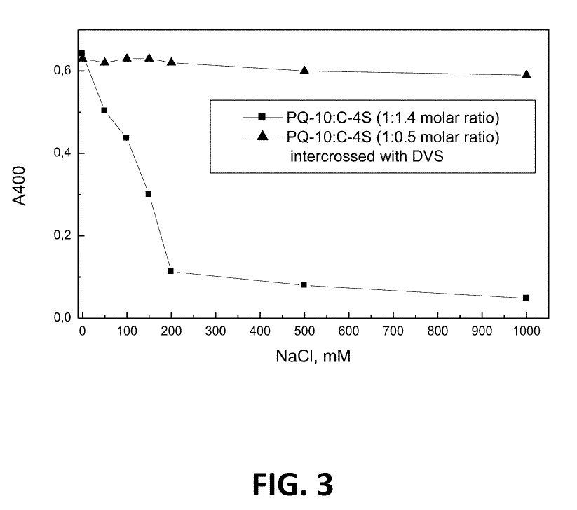

This example describes the effect of a solution with varying ionic strength on the stability of the polyquaternium-10 electrostatic complex with chondroitin 4-sulfate and the corresponding covalently cross-linked hydrogel reaction product of the reaction of polyquaternium-10 with chondroitin 4-sulfate and divinylsulfone using turbidimetric measurements at different concentrations of sodium chloride. The molar ratios of the complexes that were not covalently cross-linked were kept constant and equal to a ratio of polyquaternium-10 to chondroitin 4-sulfate of 1 M: 1.4 M, with the concentrations of sodium chloride being varied between 0 M and 1M. The molar ratios of the covalently cross-linked complexes were also kept constant and equal to a molar ratio of polyquaternium-10 to chondroitin 4-sulfate of 1 M: 0.5 M, again with the concentrations of sodium chloride being varied between 0 M and 1M. The variations in turbidity of the non-cross-linked complexes with respect to the cross-linked complexes are shown in FIG. 3. It can be seen that the turbidity of the covalently cross-linked complexes (covalently cross-linked hydrogel reaction products) did not change with changes in sodium chloride concentration, whereas, in the complexes that were not covalently cross-linked, the turbidity decreased and the interaction was totally inhibited with concentrations at 200 mM sodium chloride and above. Without being bound to theory, it is thought that this result occurs because, in the non-cross-linked complexes, the electrostatic interactions between the two polymers was weakened by the presence of charges produced by the excess of sodium chloride (NaCl) in the solution; whereas, the covalently cross-linked complexes (covalently cross-linked hydrogel reaction products) were unaffected by increases in ionic strength and their consistencies remained unchanged. Therefore, it can be concluded that the covalently cross-linked hydrogel reaction products tend to be stable in solutions with elevated ionic strength.

These tests demonstrated that the predominant interaction between the polyquaternium-10 and the chondroitin 4-sulfate is electrostatic and that when complexes containing polyquaternium-10 and the chondroitin 4-sulfate are covalently cross-linked with divinylsulfone, the resulting covalently cross-linked hydrogel reaction products are not affected by variations in ionic strength.

### Example 7: Stability of Covalently Cross-Linked and Non-Covalently-Cross-Linked Complexes to the Addition of Chondroitin 4-Sulfate

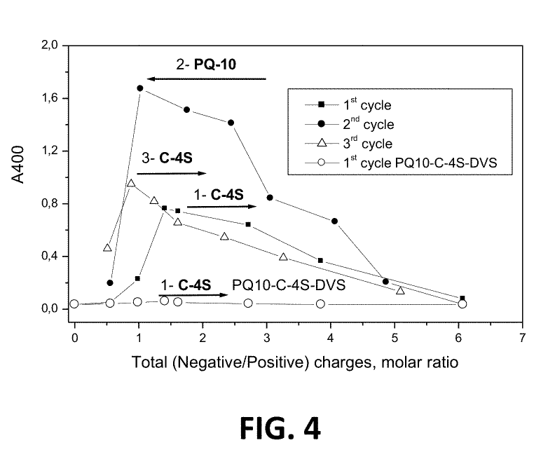

FIG. 4 shows variations in the turbidity measurements of complexes of polyquaternium-10 and chondroitin 4-sulfate that were electrostatically bound versus those that were covalently cross-linked with divinylsulfone, at different proportions of total positive/negative charge. FIG. 4 illustrates turbidity measurements at 400 nm of complexes of polyquaternium-10 with chondroitin 4-sulfate, with some complexes covalently cross-linked, as shown with open circles, and others not covalently cross-linked, shown with open triangles, wherein the charge molar ratios vary. In the first and the third cycles, the polyquaternium-10 was held constant as the chondroitin 4-sulfate was increased. Whereas in the second cycle, the chondroitin 4-sulfate was held constant as the polyquaternium-10 was increased.

For the complexes that were not covalently cross-linked, it was observed that the addition a chondroitin 4-sulfate caused an increase in the turbidity of the complex, until a molar ratio of 1 M:1.4 M of positive to negative charge was reached and precipitation of the complex occurred. Without being bound to theory, this result could be due to the excess of negative charge present in the complex, thereby causing a change of the structure where the quaternary ammonium groups are occluded inside. Further addition of the chondroitin 4-sulfate produced a gradual dissolution of the aggregates until almost complete solubilization was observed at a molar ratio of 1:6 of positive to negative charge.

A second aggregation-dissolution cycle was afterwards studied by the addition of increasing amounts of a polyquaternium-10 and by keeping constant the amount of the chondroitin 4-sulfate. In this case, it was observed that a maximum turbidity occurred at a molar ratio of 1:1 positive to negative charge. Without being bound to theory, this resultant value could be due to structural changes of polyquaternium-10 that allowed better accessibility to the cationic sites of the polymer.

Finally, this sample was subjected to a third cycle in which the same phenomenon of aggregation and dissolution was observed. Following the same procedure, seven cycles were tested qualitatively in order to probe the reversibility of the phenomenon after the third cycle. All these results revealed the presence of a cationic-anionic polyelectrolyte system with a reversible dissolution-precipitation-dissolution behavior able to occur in either direction by the addition of polycation or polyanion.

However, when the same assay was performed with complexes of the covalently cross-linked reaction of a polyquaternium-10 with a chondroitin 4-sulfate and divinylsulfone, the same absorbance all over the range and no precipitation and no redisolution of the complexes were observed. Accordingly, the covalently cross-linked hydrogel reaction product of the cross-linking reaction of a polyquaternium-10 with a chondroitin 4-sulfate and divinylsulfone exhibited greater stability than those complexes that were not covalently cross-linked.

### Example 8: Determination of Gel Content

For determination of the gel content, tests were conducted pursuant to the “American Society for Testing and Materials, Standard Test Methods for Determination of Gel Content and Swell Ratio of Cross-linked Ethylene Plastics, ASTM D2765-11, 2006” in accordance with the above described “General procedure for the determination of gel content”. Six samples were chosen from those shown in Table 4. The first three samples had a fixed concentration of polyquaternium-10 at 1% (w/v) and chondroitin 4-sulfate at 0.1% (w/v), while different initial concentrations of divinylsulfone in 1% (w/w), 3% (w/w), and 5% (w/w) based on the combined weights of the polymers in the samples, respectively. The next three samples had polyquaternium-10 at 3% (w/v) and chondroitin 4-sulfate at 0.3% (w/v), with different concentration of divinylsulfone in 1% (w/w), 3% (w/w), and 4% (w/w), respectively.

The results obtained for the selected samples are shown in Table 4:

TABLE 4
Example 8: Determination of gel content (degree of cross-linking)

| | Poly- | Chon- | | | | |
|---|---|---|---|---|---|---|
| | quaternium- | droitin | | | | Gel |
| | 10 | 4-sulfate | Divinylsulfone | M₁ | M₂ | content |
| Test^{a} | % (w/v) | % (w/w) | % (w/v) | g | g | %^{b} |
| 1-1 | 1 | 0.1 | 1 | 0.222 | 0.184 | 83 |
| 1-3 | 1 | 0.1 | 3 | 0.231 | 0.186 | 81 |
| 1-5 | 1 | 0.1 | 5 | 0.238 | 0.194 | 82 |
| 3-1 | 3 | 0.3 | 1 | 0.676 | 0.520 | 77 |
| 3-3 | 3 | 0.3 | 3 | 0.680 | 0.500 | 74 |
| 3-4 | 3 | 0.3 | 4 | 0.691 | 0.529 | 77 |

TABLE notes:
^{a}Samples combination extracted from Table 2.
^{b}Calculations carried out using Error! Reference source not found.

### Example 9: Temperature Stability Test of Covalently Cross-Linked Hydrogel Reaction Product

In this example, the stability of the covalently cross-linked hydrogel reaction products was evaluated after they were sterilized in a 15 minute autoclave sterilization cycle at 120° C. Table 5 shows the percentages of change in dynamic viscosity, storage modulus, and extrudability of samples with properties as shown in Table 2 and their preparation as specified in all combinations prepared, the cross-linked hydrogel reaction products that exhibited the greatest stability comprised the polyquaternium-10 at 3% (w/v) and 4% (w/v), the chondroitin 4-sulfate at 0.3% (w/v) and 0.4% (w/v), and divinylsulfone at 1% (w/w) and 3% (w/w) (based on the combined weights of the polymers, polyquaternium-10 and chondroitin 4-sulfate).

TABLE 5
Example 9: Stability of covalently cross-linked hydrogel reaction
products before and after the sterilization process^{a}

| | Dynamic | | | | | | | |
|---|---|---|---|---|---|---|---|---|
| | viscosity | | Storage | | Extrusion | | Storage | |
| | at 0.01 s⁻¹ | | Module | | force (g)^{b} | | conditions | |
| | (Pa) | | at 40 Hz (Pa) | | Be- | | Time, | Temp, |
| Test | Before | After | Before | After | fore | After | days^{c} | ° C.^{c} |
| 3-1 | 67640 | 3382 | 655 | 72 | 5500 | 1200 | 53 | 4 |
| 3-2 | 40389 | 10097 | 467 | 201 | 7000 | 2500 | 53 | 4 |
| 3-3 | — | 18310^{d} | — | 200^{d} | 4000 | 3500 | 53 | 4 |
| 3-4 | 26508 | 32870 | 402 | 362 | 2500 | 2000 | 16 | 4 |
| 4-3 | 54922 | 23067 | 813 | 512 | 2500 | 2500 | 16 | 4 |
| 4-4 | — | 28668^{d} | — | 749^{d} | 2000 | 2000 | 16 | 4 |

TABLE notes:
^{a}Autoclave cycle during 15 minutes at 120° C.
^{b}Needle of 27 gauge, 0.4 × 13 mm, 1 ml syringe.
^{c}Time between the production day till the moment it was submitted to autoclave.
^{d}Measured right after the autoclave cycle. The original hydrogel was very rigid and exceeded the measurement limit of the instrument.

### Example 10: Cytotoxicity Test of Covalently Cross-Linked Hydrogel Reaction Product of the Cross-Linking Reaction of Polyquaternium-10 with Chondroitin 4-Sulfate and Divinylsulfone

An in vitro test was performed on mammalian cell cultures to detect potentially cytotoxic effects using ISO elution methodology. These trials were performed by NAMSA certified laboratories (located in Northwood, Ohio, USA). The analyzed sample corresponds to test sample 3-3 of Table 2. The test was performed according to the guidelines of ISO 10993-5, “Biological evaluation of medical devices—Part 5: In vitro test for cytotoxicity”.

The tests consisted of performing a single extraction of sample 3-3 in an essential minimum medium (1×MEM) at 37° C. for 24 hours. The negative, positive and reagent controls were extracted using the same methodology. The monolayers of L-929 mouse fibroblastic cells were assayed in triplicate, each of them was dosed with each of the extracts and then incubated at 37° C. in the presence of CO₂ (5%) for 48 hours. After incubation, the monolayers were examined under a microscope at 100× magnification to evaluate cell characteristics and percentage of cellular lysis.

The sample extract showed no evidence of causing cellular lysis or toxicity. The sample extract complied with the requirements of this test because it presented a lower grade to grade 2 (mean reactivity).

### Example 11: Genotoxicity Test of Covalently Cross-Linked Hydrogel Reaction Product of the Cross-Linking Reaction of Polyquaternium-10 with Chondroitin 4-Sulfate and Divinylsulfone

Sample 3-3 of Table 2 was evaluated for its potential to cause mutagenic changes in the histidine locus of Salmonella typhimurium strains TA98, TA100, TA1535, and TA1537 or in the tryptophan locus of Eschirichia coli strain WP2uvrA. The study was performed both in the presence and absence of S9 metabolic activator based on ISO 10993-3, Biological evaluation of medical devices—Part 3: “Test for genotoxicity, carcinogenicity and reproductive toxicity” and OECD 471, guidelines for the evaluation of chemicals, reverse mutation test in bacteria. These trials were performed by NAMSA certified laboratories (located in Northwood, Ohio, USA).

The tests consisted of extracting sample 3-3 with dimethyl sulfoxide (DMSO) and in saline solution. Tubes containing molten agar were inoculated with the culture of one of the five test strains, along with the DMSO or saline extract. An aliquot of sterile water or rat liver homogenate S9 (metabolic activator) was added. The mixture was then transferred to three petri dishes. A parallel test was performed for the negative control (vehicle extract only) and positive controls. The mean reverting colony of the sample was compared with the mean of negative control colonies of the negative control for each of the strains.

Both extracts, in DMSO and in saline, were considered to be non-mutagenic to S. typhimurium strains tested TA98, TA100, TA1535, and TA1537 and to E. coli strain of WP2uvrA test.

### Example 12: Sensibilization Test of Covalently Cross-Linked Hydrogel Reaction Product of the Cross-Linking Reaction of Polyquaternium-10 with Chondroitin 4-Sulfate and Divinylsulfone

Sample 3-3 of Table 2 was evaluated for its potential to cause delayed sensitization by dermal contact in a guinea-pig maximization test. This study was performed in NAMSA certified laboratories (located in Northwood, Ohio, USA) and according to the requirements of ISO 10993-10, “Biological evaluation of medical devices—Part 10: Dermal irritation and sensitization test”.

The tests consisted of extracting sample 3-3 with a solution of 0.9% sodium chloride USP and in sesame oil, NF. Each extract was injected intradermically followed by the placement of an occlusive patch into 10 guinea pigs (per extract). The extraction vehicle was injected in the same manner with subsequent patch placement to five control guinea pigs (per extract). After the recovery period, the study and control animals received an additional patch with the corresponding extract and control vehicle. All sites were scored according to the dermal reaction observed at 24 and 48 hours after patch removal.

Extracts from the samples showed no evidence of delayed sensitization to dermal contact in guinea pigs. Therefore, the sample was not considered as a sensitizer in the guinea-pig maximization test.

### Example 13: Evaluation of Different Radiopaque Agents to Visualize Implantation of the Covalently Cross-Linked Hydrogel Reaction Product of the Cross-Linking Reaction of Polyquaternium-10 with Chondroitin 4-Sulfate and Divinylsulfone

Different radiopaque substances were tested and mixed with the covalently cross-linked hydrogel reaction product of the cross-linking reaction of polyquaternium-10 with chondroitin 4-sulfate and divinylsulfone to evaluate their opacity in X-ray studies, in order to obtain the best contrasting formulation for a better monitoring of the in vivo formulation. The radiopaque substances tested were triyosom C (a commercial tri-iodinated organic compound), MnO₂, and BaCl₂.

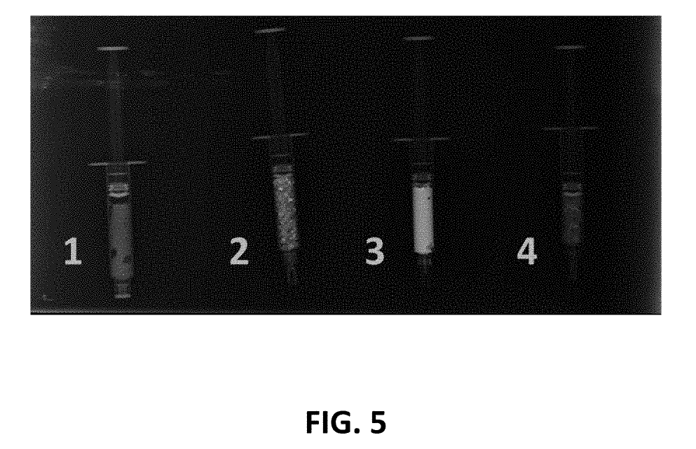

FIG. 5 shows the radiographs obtained for each formulation, compared to a hydrogel sample without radiopaque substance. The formulation with the best radiopacity is hydrogel-triyosom (lane 3 of the figure). Radiopacity of prefilled syringes: syringe (1) includes BaCl₂ mixed with a hydrogel made from a reaction mixture comprising 3% (w/v) polyquaternium-10, 0.3% (w/v) chondroitin 4-sulfate, and 3% (w/w) divinylsulfone, based on the combined weights of the polymers; syringe (2) includes 15% (w/w) MnO₂, based on the weight of the hydrogel, mixed with a hydrogel made from a reaction mixture comprising 3% (w/v) polyquatemium-10, 0.3% (w/v) chondroitin 4-sulfate, and 3% (w/w) divinylsulfone, based on the combined weights of the polymers; syringe (3) includes 20% (w/w) triyosom, based on the weight of the hydrogel, mixed with a hydrogel made from a reaction mixture comprising 3% (w/v) polyquatemium-10, 0.3% (w/v) chondroitin 4-sulfate, and 3% (w/v) divinylsulfone, based on the weight of the polymers; and, syringe (4) includes a hydrogel made from a reaction mixture comprising 3% (w/v) polyquatemium-10, 0.3% (w/v) chondroitin 4-sulfate, and 3% (w/v) divinylsulfone, based on the weight of the polymers.

### Example 14: Radiological Evaluation after Implantation in a Pig Knee Joint of the Covalently Cross-Linked Hydrogel Reaction Product of the Cross-Linking Reaction of Polyquatemium-10 with Chondroitin 4-Sulfate and Divinylsulfone Mixed with Triyosom

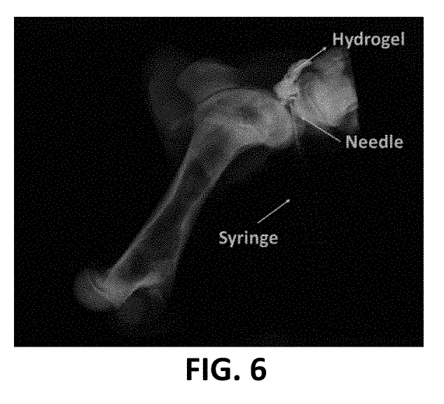

FIG. 6 shows a radiograph of pig knee joint implanted with a mixture of 40% (w/w) triyosom C, based on the weight of the hydrogel, and a hydrogel made from a reaction mixture comprising 3% (w/v) of polyquaternium-10, 0.3% (w/v) of chondroitin 4-sulfate, and 3% (w/v) of divinylsulfone, based on the combined weight of the polymers. The radiograph was made in order to detect distribution of the hydrogel in the joint after injection into the intra-articular space. After injection of the hydrogel, movement of the knee was made to produce homogeneous distribution of the hydrogel in the intra-articular space.

### Example 15: Implantation in Rabbits' Knees of the Covalently Cross-Linked Hydrogel Reaction Product of the Cross-Linking Reaction of Polyquaternium-10 with Chondroitin 4-Sulfate and Divinylsulfone, Wherein the Rabbits' Knees Exhibit Induced Osteoarthritis

Sample 3-3 of Table 2 was implanted in rabbits to evaluate safety of the substance for the treatment of osteoarthritis in the knee. This study was carried out in the certified laboratories of the Center for Comparative Medicine (CMC), Universidad Nacional del Littoral-CONICET, Esperanza, Santa Fe, Argentina. The study included performing an evaluation of the safety using an osteoarthritis model generated by the transection of the anterior cruciate ligament in the knee joint in New Zealand strain rabbits. Young adult males and females were used, with a mean weight of 3±0.5 kg for males and 3.5±0.5 kg for females. Two experimental groups of 18 animals were designed for each group (9 males and 9 females) with different completion points at 3, 6 and 12 months. The transaction of each test was performed in the right knee with the left knee untreated to act as a sham control. At 28 days after transection of the cruciate ligament, 0.3 mL of sample 3-3 was injected into the group A in both knees and 0.3 mL of saline (placebo) was injected into the group B in both knees.

A study of biochemical and hematological variables and a radiological study before the start, at 28 days, and at 3, 6 and 12 months after surgery were performed. Euthanasia and complete necropsy were performed at 3, 6 and 12 months after surgery. In addition, a histological study of the site of implantation and control, inguinal lymph nodes, liver and spleen were carried out. Microscopic examination of the joint determined the presence of inflammatory components, neovascularization, fibrosis, necrosis and any finding that was considered histologically relevant.

After the tests were conducted, it was found that no significant effects on either sex or treatment on weight variation occurred. Also, effects associated with sex were observed in some hematological variables, but no differences attributable to treatments were found. There were no differences in the biochemical variables attributable to the treatments, nor the sex, nor any interaction between the two. Additionally, in the histopathological study of the articular capsules, no differences were detected in the analyzed variables between the right and left knees of each group at each sampling point. However, comparing the variables analyzed in each knee between groups revealed significant trends and differences in the proliferation of blood vessels, fibroblasts/fibrocytes and synoviocytes in the placebo group compared to group A. Furthermore, in the histological analysis of the femurs, between the two knees, there were no differences. Based on the information collected, it can be concluded that the formulations were harmless since no biochemical or hematological parameters were altered in association with the treatment, demonstrating systemic safety at the dose used.

### Example 16: Implantation in Rabbit's Knee Joints with Induced Osteoarthritis of the Covalently Cross-Linked Hydrogel Reaction Product of the Cross-Linking Reaction of Polyquaternium-10 with Chondroitin 4-Sulfate and Divinylsulfone and Comparison with SYNVISC ONE® (Hylan G-F 20)

A comparative study between the covalently cross-linked hydrogel reaction product of the cross-linking reaction of polyquaternium-10 with chondroitin 4-sulfate and divinylsulfone, sample 3-3 of Table 2, and SYNVISC ONE® (hylan G-F 20) was carried out in the certified laboratories of the Center for Comparative Medicine (CMC), Universidad Nacional del Littoral-CONiCET, Esperanza, Santa Fe, Argentina. The study consisted of osteoarthritis model generated by the transection of the anterior cruciate ligament in the knee joint in New Zealand strain rabbits. Young adult males and females were used, with a mean weight of 3±0.5 kg for males and 3.5±0.5 kg for females, twelve animals were designed (six males and six females) with different completion points at 6 and 12 months. Transaction was performed in both knee joints of each rabbit, at 28 days after transection 0.3 mL of sample 3-3 was injected into right knee and 0.3 mL of SYNVISC ONE® (hylan G-F 20) was injected in the left knee joint.

A study of the biochemical and the hematological variables and a radiological study before the start, at 28 days, and at 6 and 12 months after surgery were performed. Euthanasia and complete necropsy were performed at 6 and 12 months after surgery. In addition, a histological study of the site of implantation and control, inguinal lymph nodes, liver and spleen will be carried out. Microscopic examination of the joint determines the presence of inflammatory components, neovascularization, fibrosis, necrosis and any finding that is considered histologically relevant.

Macroscopic study of cartilage damage was performed and analyzed using the India ink technique described by Yoshioka M, Coutts R D, Amiel D, Hacker S A in “Characterization of a model of osteoarthritis in the rabbit knee. Osteoarthritis and Cartilage” (1996 June; 4(2):87-98). End point at 6 months did not reveal significant trends or differences. Nevertheless, at end point 12 months, significant differences in the cartilage damage were revealed. A severe damage was observed for the knee joint implanted with SYNVISC ONE® (hylan G-F 20), while the knee joint with the covalently cross-linked hydrogel reaction product of the cross-linking reaction of polyquaternium-10 with chondroitin 4-sulfate and divinylsulfone showed only mild cartilage damage. While not being bound to theory, it is possible that this result could be attributable to differences in stability between the two materials. SYNVISC ONE® (hylan G-F 20) is known to have a stability of 6-8 months once implanted. The covalently cross-linked hydrogel reaction product of the cross-linking reaction of polyquaternium-10 with chondroitin 4-sulfate and divinylsulfone appears to be more stable and, therefore, appears to slow the progression of cartilage damage.

Histopathological evaluation of the femurs showed significant differences between the right and left knees. Less damage was observed in the right knee, which was injected with the covalently cross-linked hydrogel reaction product of the cross-linking reaction of polyquaternium-10 with chondroitin 4-sulfate and divinylsulfone.

### Example 17: Cadaveric Study: Determination of Injection and Behavior of the Covalently Cross-Linked Hydrogel Reaction Product of the Cross-Linking Reaction of Polyquaternium-10 with Chondroitin 4-Sulfate and Divinylsulfone and Study of the Usability of Product

An amount of sample 3-3 of Table 2 by itself and another amount of sample 3-3 mixed with a contrasting agent of triyosom C at 40% (v/v) were used in a cadaveric study in order to visualize injection of the hydrogel into articular space and perform usability of the product. The hydrogel materials were installed in polycarbonate syringes of 1 ml and 10 ml volumes and injected into the knees of cadaver specimens simulating viscosupplementation technique. The injection was visualized and guided by fluoroscopy. This study was carried out in the certified laboratories of the Surgical Anatomy Training Center by Simulators (CEAQUS), Maimónides University, Buenos Aires, Argentina.

Selected approaches to knee joint injection were paths of least obstruction and maximal access to the synovial cavity and included, alternatively, superolateral, superomedial, or anteromedial/anterolateral injection. The knee joint injection site was selected according to the subject's bony anatomy and was marked with the tip of a retracted ballpoint pen before injection. While the procedure could include removal of any synovial fluid (effusion) using a 18-20 gauge needle, if conducted in a living subject, in cadaveric study effusion of synovial fluid was not necessary and was not conducted. Injection was performed using standard technique and using needles of 18-23 gauge. Visualization of injection was followed by fluoroscopy. Avoidance of extra-articular injection of hydrogel, injection into the synovial tissues, injection into the fat pad or joint capsule, or intravascular injection was taken into account.

After the injection, flexion and bending of the articulation was performed 3-6 times following injection in order to allowed the hydrogel to distribute into the articulation. The study was conducted according to IEC 62366 in order to evaluate the usability of the kit (syringes filled with hydrogel and needles). It was determined that the hydrogel could be efficiently manually injected into the intra-articular space using 18-23 gauge needles. No effusion of the hydrogel from the articular capsule was observed when bending the articulation.

### Example 18: Characterization of the Covalently Cross-Linked Hydrogel Reaction Product of the Cross-Linking Reaction of Polyquaternium-10 with Chondroitin 4-Sulfate and Divinylsulfone by Differential Scanning Calorimetry (DSC), Thermogravimetric Analysis (TGA) and pH Determination

The hydrogel content of one syringe with a volume of 6 ml was lyophilized during 48 hours. A syringe containing the non-lyophilized hydrogel was defined as Sample C, while the lyophilized hydrogel in the other syringe was defined as Sample D. Both samples were stored at room temperature in a closed container.

Analysis of the thermal behavior of the samples was carried out via thermogravimetric analysis (TGA) using a SHIMADZU® TGA-50 Thermogravimetric analyzer. The samples were heated at a rate of 10° C. per minute from a starting temperature of 23° C. to a final temperature of 600° C. in an atmosphere of nitrogen 5.0, using nickel standard (Curie temperature) calibration.

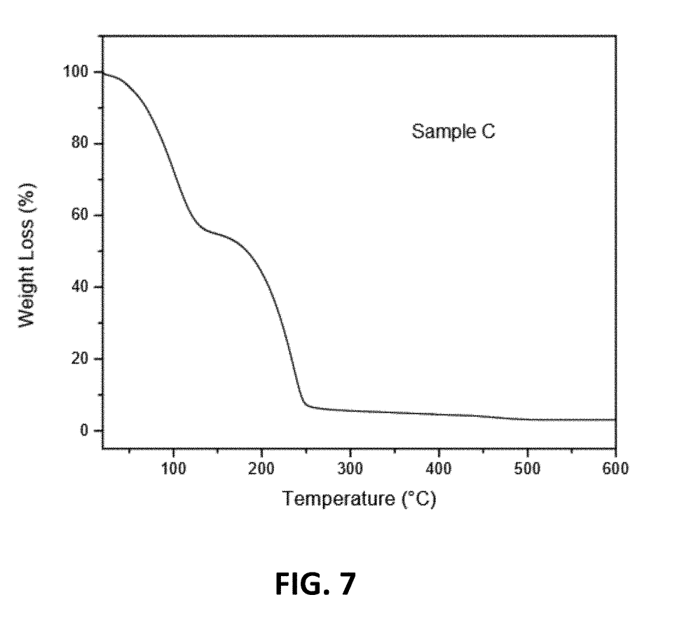

FIG. 7 shows the thermogram generated in the analysis of the covalently cross-linked hydrogel reaction product of the cross-linking reaction of polyquaternium-10 with chondroitin 4-sulfate and divinylsulfone. The starting temperature of the transition or onset temperature (Tₒ) was determined by extrapolating the slopes before and after the transition. As shown in FIG. 7, at temperatures below 150° C., a 45% loss of mass associated with removal of water from the hydrogel material (Tₒ=62.5° C.) was observed. From this temperature up to 600° C., a thermal event was observed associated with the decomposition of the hydrogel material. Sample C presented a Tₒ of 201.2° C. with a loss of mass of 49.4%. The total mass loss at 600° C. led to a residue of 3.0% for sample C.

Calorimetric thermal analysis of the samples was conducted via differential scanning calorimetry (DSC) using a PERKINELMER® Pyris 1 calorimeter. The samples were subjected to a nitrogen atmosphere using an Indian standard calibration and an aluminum capsule material. The samples were subjected to a first heating cycle in which they were heated at a rate of 10° C. per minute from an initial temperature of −35° C. to a final temperature of 200° C. The samples were then subjected to a cooling cycle at rate of 10° C. per minute from an initial temperature of 200° C. back to a final temperature of −35° C. The samples were then subjected to a second heating cycle at a rate of 10° C. per minute from an initial temperature of −35° C. to a final temperature of 200° C.

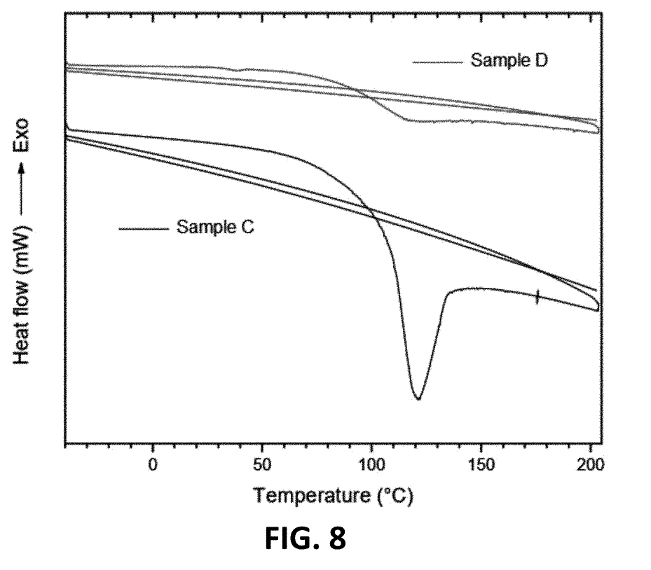

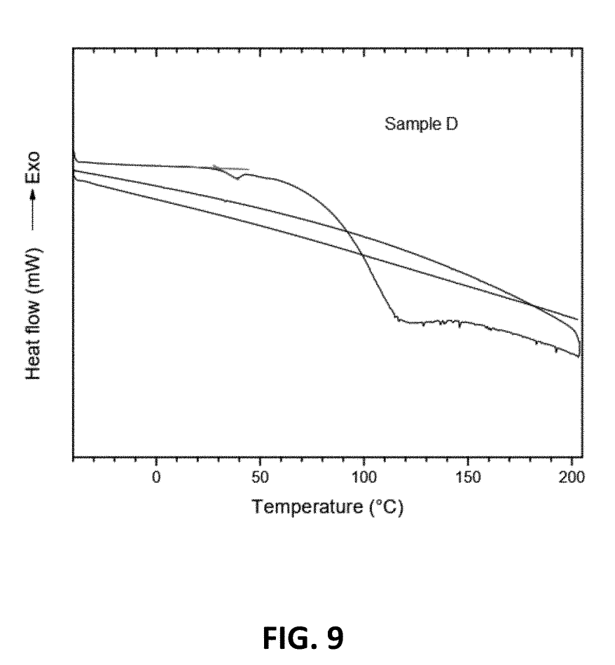

FIG. 8 shows the thermograms corresponding to the heating/cooling/heating scans of sample C hydrogel and sample D lyophilized product. FIG. 9 shows an expanded view of the thermal response of the sample D lyophilized product.

In FIG. 8, both samples presented an endothermic event around 100° C. due to the evaporation of water, but much more pronounced for the sample C hydrogel. The loss of water in sample D indicated the presence of water strongly associated by hydrogen bonding to the macromolecular chains. The lyophilized sample D hydrogel was studied by this technique because no glass transition temperature (Tg) was observed in the sample C hydrogel, possibly because the large amount of water masked the thermal events of the macromolecules. However, for sample D it was possible to observe a softening temperature (Tg), glass transition temperature) at 29.9° C. As shown in both FIGS. 8 and 9, the cooling and second heating stages for each sample did not show the occurrence of any transition.

The sample of the covalently cross-linked hydrogel reaction product of the cross-linking reaction of polyquaternium-10 with chondroitin 4-sulfate and divinylsulfone presented two thermal events of mass loss, the first associated with water loss, and the second due to degradation of the material starting at around a temperature greater than about 150° C. The sample C hydrogel was amorphous, and it was only possible to observe a glass transition for the lyophilized sample (Tg=29.9° C.).

### Example 19: Determination of pH Value of the Covalently Cross-Linked Hydrogel Reaction Product of the Cross-Linking Reaction of Polyquaternium-10 with Chondroitin 4-Sulfate and Divinylsulfone

The pH of the sample C hydrogel was tested via pH-metry using a Lutron Model 208 pH meter. The pH meter was calibrated with commercial buffer solutions (pH=4, Anedra, Batch 22426-2 and pH=7, Biopack, Lot 9352013). Sample C was studied in equilibrium conditions in MILLI-Q® deionized water. The measurements were made in triplicate at 25±2° C. The pH value of the MILLI-Q® deionized water was 6.29, while the value obtained for sample C was a pH of 6.15±0.08. Therefore, the hydrogel sample does not produce a significant variation in the pH value of the aqueous medium under the conditions that were studied.

In the following, various aspects of the present disclosure are described with reference to numbered paragraphs:

1. A composition comprising:

a covalently cross-linked hydrogel reaction product of a cross-linking reaction of a reaction mixture comprising a polyquaternium-10, a chondroitin 4-sulfate, and a divinylsulfone, and wherein the covalently cross-linked hydrogel reaction product exhibits a gel content in the range of 65% to 85% as determined by ASTM D2765-11, 2006.

2. The composition of paragraph 1, wherein the covalently cross-linked hydrogel reaction product exhibits a gel content in the range of 70% to 80% as determined by ASTM D2765-11, 2006.
3. The composition of paragraph 1, wherein the covalently cross-linked hydrogel reaction product exhibits a gel content in the range of 72% to 78% as determined by ASTM D2765-11, 2006.
4. The composition of any of paragraphs 1 to 3, wherein the reaction mixture comprises the chondroitin 4-sulfate in a range of 0.1% (w/v) to 1.2% (w/v) based on volume of the reaction mixture.
5. The composition of any of paragraphs 1 to 3, wherein the reaction mixture comprises the chondroitin 4-sulfate in a range of 0.2% (w/v) to 0.3% (w/v) based on volume of the reaction mixture.
6. The composition of any of paragraphs 1 to 5, wherein the reaction mixture comprises the polyquaternium-10 in a range of 1% (w/v) to 4% (w/v) based on volume of the reaction mixture.
7. The composition of any of paragraphs 1 to 5, wherein the reaction mixture comprises the polyquaternium-10 in a range of 2% (w/v) to 3% (w/v) based on volume of the reaction mixture.
8. The composition of any of paragraphs 1 to 7, wherein the reaction mixture comprises the divinylsulfone in a range of 1% (w/w) to 4% (w/w) based on combined weights of the polyquaternium-10 and the chondroitin 4-sulfate in the reaction mixture.
9. The composition of any of paragraphs 1 to 7, wherein the reaction mixture comprises the divinylsulfone in a range of 2% (w/w) to 3% (w/w) based on combined weights of the polyquternium-10 and the chondroitin 4-sulfate in the reaction mixture.
10. The composition of any of paragraphs 1 to 9, wherein the reaction mixture comprises the polyquaternium-10 and the chondroitin 4-sulfate in a range of weight ratios of 10:1 to 10:3 based on weights of the polyquaternium-10 and the chondroitin 4-sulfate in the reaction mixture.
11. The composition of any of paragraphs 1 to 10, further comprising an isobutylphenylpropionic acid.
12. The composition of any of paragraphs 1 to 11, further comprising a Sodium; 2-[2-(2,6-dichloroanilino)phenyl]acetate.
13. The composition of any of paragraphs 1 to 12, wherein the covalently cross-linked hydrogel reaction product exhibits a dynamic viscosity at a shear rate of 0.01/s in the range of 3000 Pa to 33000 Pa as determined by ASTM D2084-95, 1994.
14. The composition of any of paragraphs 1 to 12, wherein the covalently cross-linked hydrogel reaction product exhibits a dynamic viscosity at a shear rate of 0.01/s in the range of 10000 to 24000 Pa as determined by ASTM D2084-95, 1994.
15. The composition of any of paragraphs 1 to 12, wherein the covalently cross-linked hydrogel reaction product exhibits a dynamic viscosity at a shear rate of 0.01/s in the range of 15000 to 20000 Pa as determined by ASTM D2084-95, 1994.
16. The composition of any of paragraphs 1 to 15, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.
17. A method of producing the covalently cross-linked hydrogel reaction product of the composition of any of paragraphs 1 to 16, comprising:

combining a first aqueous solution comprising the chondroitin 4-sulfate with a second aqueous solution comprising the polyquaternium-10 to form an aqueous mixture;

adding an alkaline solution to the aqueous mixture to form an alkaline aqueous mixture;

adding a third aqueous solution comprising the divinylsulfone to the alkaline aqueous mixture to form the reaction mixture;

allowing a covalent cross-linking reaction to occur in the reaction mixture to form an intermediate reaction product;

neutralizing the intermediate reaction product;

washing the intermediate reaction product with a buffered solution;

filtering the intermediate reaction product; and,

adjusting pH of the intermediate reaction product to form the covalently cross-linked hydrogel reaction product.

18. The method of paragraph 17, wherein the first aqueous solution comprises the chondroitin 4-sulfate in a range of 0.6% (w/v) to 8.0% (w/v) based on volume of the first aqueous solution.
19. The method of paragraph 17, wherein the first aqueous solution comprises the chondroitin 4-sulfate in a range of 1.2% (w/v) to 4.0% (w/v) based on volume of the first aqueous solution.
20. The method of paragraph 17, wherein the first aqueous solution comprises the chondroitin 4-sulfate in a range of 1.6% (w/v) to 2.0% (w/v) based on volume of the first aqueous solution.
21. The method of any of paragraphs 17 to 20, wherein the second aqueous solution comprises the polyquatemium-10 in a range of 1.4% (w/v) to 5.7% (w/v) based on volume of the second aqueous solution.
22. The method of any of paragraph 17 to 20, wherein the second aqueous solution comprises the polyquatemium-10 in a range of 3.7% (w/v) to 4.8% (w/v) based on volume of the second aqueous solution.
23. The method of any of paragraphs 17 to 20, wherein the second aqueous solution comprises the polyquatemium-10 in a range of 4.2% (w/v) to 4.4% (w/v) based on volume of the second aqueous solution.
24. The method of any of paragraphs 17 to 23, wherein the third aqueous solution comprises the divinylsulfone in a range of 1.2% (w/v) to 4.8% (w/v) based on volume of the third aqueous solution.
25. The method of any of paragraphs 17 to 23, wherein the third aqueous solution comprises the divinylsulfone in a range of 2% (w/v) to 4% (w/v) based on volume of the third aqueous solution.
26. The method of any of paragraphs 17 to 23, wherein the third aqueous solution comprises the divinylsulfone in a range of 2.8% (w/v) to 3.2% (w/v) based on the volume of the third aqueous solution.
27. The method of any of paragraphs 17 to 26, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.
28. The composition of any of paragraphs 1 to 16 for use in the treatment of osteoarthritis.
29. The composition for use of paragraph 28, wherein the composition is in a form for administration by injection into a joint of a subject.
30. The composition for use of paragraph 29, wherein the composition is in a form for administration by injection into the joint in an amount in the range of 1 ml to 10 ml.
31. A kit comprising:

the composition of any of paragraphs 1 to 16, and a syringe for injecting said composition into a joint.

32. The kit of paragraph 31, further comprising a needle.
33. A composition comprising:

a covalently cross-linked hydrogel reaction product of a cross-linking reaction of a reaction mixture comprising a polyquaternium-10, a chondroitin 4-sulfate, and a divinylsulfone, wherein the reaction mixture comprises the chondroitin 4-sulfate in a range of 0.2% (w/v) to 0.3% (w/v) based on volume of the reaction mixture, wherein the reaction mixture comprises the polyquaternium-10 in a range of 2% (w/v) to 3% (w/v) based on volume of the reaction mixture, wherein the reaction mixture comprises the divinylsulfone in a range of 2% (w/w) to 3% (w/w) based on combined weights of the polyquternium-10 and the chondroitin 4-sulfate in the reaction mixture, wherein the chondroitin 4-sulfate, the polyquaternium-10, and the divinylsulfone are covalently cross-linked in the covalently cross-linked hydrogel reaction product, and wherein the covalently cross-linked hydrogel reaction product exhibits a gel content in the range of 72% to 78% as determined by ASTM D2765-11, 2006.

34. The composition of paragraph 33, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.
35. A method of producing the covalently cross-linked hydrogel reaction product of the composition of any of paragraphs 33 or 34, comprising:

combining a first aqueous solution comprising the chondroitin 4-sulfate with a second aqueous solution comprising the polyquaternium-10 to form an aqueous mixture, wherein the first aqueous solution comprises the chondroitin 4-sulfate in a range of 1.6% (w/v) to 2.0% (w/v) based on volume of the first aqueous solution, and wherein the second aqueous solution comprises the polyquaternium-10 in a range of 4.2% (w/v) to 4.4% (w/v) based on volume of the second aqueous solution;

adding an alkaline solution to the aqueous mixture to form an alkaline aqueous mixture;

adding a third aqueous solution comprising the divinylsulfone to the alkaline aqueous mixture to form the reaction mixture, wherein the third aqueous solution comprises the divinylsulfone in a range of 2.8% (w/v) to 3.2% (w/v) based on the volume of the third aqueous solution;

allowing a covalent cross-linking reaction to occur in the reaction mixture to form an intermediate reaction product;

neutralizing the intermediate reaction product to form a neutralized intermediate reaction product;

washing the neutralized intermediate reaction product with a buffered solution to form a washed neutralized intermediate reaction product;

filtering the washed neutralized intermediate reaction product to form a filtered washed neutralized intermediate reaction product; and, adjusting pH of the filtered washed neutralized intermediate reaction product to form the covalently cross-linked hydrogel reaction product.

36. The composition of any of paragraphs 33 or 34 for use in the treatment of osteoarthritis.
37. The composition for use of paragraph 36, wherein the composition is in a form for administration by injection into a joint of a subject.
38. A kit comprising:

the composition of paragraphs 33 or 34, and a syringe for injecting said composition into a joint.

It will be apparent to those skilled in the art that various modifications and variations can be made to the compositions, hydrogels, hydrogel reaction products, methods of production, methods of use, kits, and systems set forth herein and are contemplated by the present disclosure.

1. A composition comprising:
a covalently cross-linked hydrogel reaction product of a cross-linking reaction of a reaction mixture comprising a polyquaternium-10, a chondroitin 4-sulfate, and a divinylsulfone, and wherein the covalently cross-linked hydrogel reaction product exhibits a gel content in the range of 65% to 85% as determined by ASTM D2765-11, 2006.

2. The composition of claim 1, wherein the covalently cross-linked hydrogel reaction product exhibits a gel content in the range of 70% to 80% as determined by ASTM D2765-11, 2006.

3. The composition of claim 1, wherein the covalently cross-linked hydrogel reaction product exhibits a gel content in the range of 72% to 78% as determined by ASTM D2765-11, 2006.

4. The composition of claim 1, wherein the reaction mixture comprises the chondroitin 4-sulfate in a range of 0.1% (w/v) to 1.2% (w/v) based on volume of the reaction mixture.

5. The composition of claim 1, wherein the reaction mixture comprises the chondroitin 4-sulfate in a range of 0.2% (w/v) to 0.3% (w/v) based on volume of the reaction mixture.

6. The composition of claim 1, wherein the reaction mixture comprises the polyquaternium-10 in a range of 1% (w/v) to 4% (w/v) based on volume of the reaction mixture.

7. The composition of claim 1, wherein the reaction mixture comprises the polyquaternium-10 in a range of 2% (w/v) to 3% (w/v) based on volume of the reaction mixture.

8. The composition of claim 1, wherein the reaction mixture comprises the divinylsulfone in a range of 1% (w/w) to 4% (w/w) based on combined weights of the polyquaternium-10 and the chondroitin 4-sulfate in the reaction mixture.

9. The composition of claim 7, wherein the reaction mixture comprises the divinylsulfone in a range of 2% (w/w) to 3% (w/w) based on combined weights of the polyquternium-10 and the chondroitin 4-sulfate in the reaction mixture.

10. The composition of claim 1, wherein the reaction mixture comprises the polyquaternium-10 and the chondroitin 4-sulfate in a range of weight ratios of 10:1 to 10:3 based on weights of the polyquaternium-10 and the chondroitin 4-sulfate in the reaction mixture.

11. The composition of claim 1, further comprising an isobutylphenylpropionic acid.

12. The composition of claim 1, further comprising a Sodium; 2-[2-(2,6-dichloroanilino)phenyl]acetate.

13. The composition of claim 1, wherein the covalently cross-linked hydrogel reaction product exhibits a dynamic viscosity at a shear rate of 0.01/s in the range of 3000 Pa to 33000 Pa as determined by ASTM D2084-95, 1994.

14. The composition of claim 1, wherein the covalently cross-linked hydrogel reaction product exhibits a dynamic viscosity at a shear rate of 0.01/s in the range of 10000 to 24000 Pa as determined by ASTM D2084-95, 1994.

15. The composition of claim 1, wherein the covalently cross-linked hydrogel reaction product exhibits a dynamic viscosity at a shear rate of 0.01/s in the range of 15000 to 20000 Pa as determined by ASTM D2084-95, 1994.

16. The composition of claim 1, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.

17. A method of producing the covalently cross-linked hydrogel reaction product of the composition of claim 1, comprising:
combining a first aqueous solution comprising the chondroitin 4-sulfate with a second aqueous solution comprising the polyquaternium-10 to form an aqueous mixture;
adding an alkaline solution to the aqueous mixture to form an alkaline aqueous mixture;
adding a third aqueous solution comprising the divinylsulfone to the alkaline aqueous mixture to form the reaction mixture;
allowing a covalent cross-linking reaction to occur in the reaction mixture to form an intermediate reaction product;
neutralizing the intermediate reaction product;
washing the intermediate reaction product with a buffered solution;
filtering the intermediate reaction product; and,
adjusting pH of the intermediate reaction product to form the covalently cross-linked hydrogel reaction product of the composition of claim 1.

18. The method of claim 17, wherein the first aqueous solution comprises the chondroitin 4-sulfate in a range of 0.6% (w/v) to 8.0% (w/v) based on volume of the first aqueous solution.

19. The method of claim 17, wherein the first aqueous solution comprises the chondroitin 4-sulfate in a range of 1.2% (w/v) to 4.0% (w/v) based on volume of the first aqueous solution.

20. The method of claim 17, wherein the first aqueous solution comprises the chondroitin 4-sulfate in a range of 1.6% (w/v) to 2.0% (w/v) based on volume of the first aqueous solution.

21. The method of claim 17, wherein the second aqueous solution comprises the polyquaternium-10 in a range of 1.4% (w/v) to 5.7% (w/v) based on volume of the second aqueous solution.

22. The method of claim 17, wherein the second aqueous solution comprises the polyquaternium-10 in a range of 3.7% (w/v) to 4.8% (w/v) based on volume of the second aqueous solution.

23. The method of claim 17, wherein the second aqueous solution comprises the polyquaternium-10 in a range of 4.2% (w/v) to 4.4% (w/v) based on volume of the second aqueous solution.

24. The method of claim 17, wherein the third aqueous solution comprises the divinylsulfone in a range of 1.2% (w/v) to 4.8% (w/v) based on volume of the third aqueous solution.

25. The method of claim 17, wherein the third aqueous solution comprises the divinylsulfone in a range of 2% (w/v) to 4% (w/v) based on volume of the third aqueous solution.

26. The method of claim 17, wherein the third aqueous solution comprises the divinylsulfone in a range of 2.8% (w/v) to 3.2% (w/v) based on the volume of the third aqueous solution.

27. The method of claim 17, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.

28. A method of treating a joint of a subject comprising:
injecting into the joint the composition of claim 1.

29. The method of claim 28, wherein the composition of claim 1 is injected into the joint in an amount in the range of 1 ml to 10 ml.

30. A kit for treating joints comprising:
the composition of claim 1, and a syringe for injecting the composition of claim 1 into a joint.

31. The kit of claim 30, further comprising a needle.

32. A composition comprising:
a covalently cross-linked hydrogel reaction product of a cross-linking reaction of a reaction mixture comprising a polyquaternium-10, a chondroitin 4-sulfate, and a divinylsulfone, wherein the reaction mixture comprises the chondroitin 4-sulfate in a range of 0.2% (w/v) to 0.3% (w/v) based on volume of the reaction mixture, wherein the reaction mixture comprises the polyquaternium-10 in a range of 2% (w/v) to 3% (w/v) based on volume of the reaction mixture, wherein the reaction mixture comprises the divinylsulfone in a range of 2% (w/w) to 3% (w/w) based on combined weights of the polyquternium-10 and the chondroitin 4-sulfate in the reaction mixture, wherein the chondroitin 4-sulfate, the polyquaternium-10, and the divinylsulfone are covalently cross-linked in the covalently cross-linked hydrogel reaction product, and wherein the covalently cross-linked hydrogel reaction product exhibits a gel content in the range of 72% to 78% as determined by ASTM D2765-11, 2006.

33. The composition of claim 32, wherein the reaction mixture comprises 3% (w/v) of polyquaternium-10 based on the volume of the reaction mixture, 0.3% (w/v) chondroitin 4-sulfate based on the volume of the reaction mixture, and 3% (w/w) divinylsulfone based on the combined weights of the polyquaternium-10 and the chondroitin 4-sulfate in the reaction mixture, and wherein the covalently cross-linked hydrogel reaction product comprises 46.2% by weight C, 10.5% by weight H, 40.3% by weight O, 1.6% by weight N, and 1.4% by weight S.

34. A method of producing the covalently cross-linked hydrogel reaction product of the composition of claim 32, comprising:
combining a first aqueous solution comprising the chondroitin 4-sulfate with a second aqueous solution comprising the polyquaternium-10 to form an aqueous mixture, wherein the first aqueous solution comprises the chondroitin 4-sulfate in a range of 1.6% (w/v) to 2.0% (w/v) based on volume of the first aqueous solution, and wherein the second aqueous solution comprises the polyquaternium-10 in a range of 4.2% (w/v) to 4.4% (w/v) based on volume of the second aqueous solution;
adding an alkaline solution to the aqueous mixture to form an alkaline aqueous mixture;
adding a third aqueous solution comprising the divinylsulfone to the alkaline aqueous mixture to form the reaction mixture, wherein the third aqueous solution comprises the divinylsulfone in a range of 2.8% (w/v) to 3.2% (w/v) based on the volume of the third aqueous solution;
allowing a covalent cross-linking reaction to occur in the reaction mixture to form an intermediate reaction product;
neutralizing the intermediate reaction product to form a neutralized intermediate reaction product;
washing the neutralized intermediate reaction product with a buffered solution to form a washed neutralized intermediate reaction product;
filtering the washed neutralized intermediate reaction product to form a filtered washed neutralized intermediate reaction product; and,
adjusting pH of the filtered washed neutralized intermediate reaction product to form the covalently cross-linked hydrogel reaction product of the composition of claim 32.

35. A method of treating a joint of a subject comprising:
injecting into the joint the composition of claim 32.

36. A kit for treating joints comprising:
the composition of claim 32, and a syringe for injecting the composition of claim 32 into a joint.
